(19) United States
(12) Patent Application Publication
(10) Pub. No.: US 2025/0127656 A1
(43) Pub. Date: Apr. 24, 2025

(54) HEATING AND MICROCLIMATE MANAGEMENT SYSTEM FOR PATIENT SUPPORT APPARATUS

(71) Applicant: Hill-Rom Services, Inc., Batesville, IN (US)

(72) Inventors: Jnanesha Ramegowda, Batesville, IN (US); Olivia Gubitose Boyce, Cincinnati, OH (US)

(21) Appl. No.: 18/915,837

(22) Filed: Oct. 15, 2024

Related U.S. Application Data
(60) Provisional application No. 63/591,519, filed on Oct. 19, 2023.

(51) Int. Cl. A61F 7/08; A61F 7/00; A61G 7/065

(52) U.S. Cl. CPC A61F 7/08; A61F 7/0085; A61F 7/0097; A61G 7/065; A61F 2007/006; A61F 2007/0064; A61F 2007/0072; A61F 2007/0093; A61F 2007/0096

(57) ABSTRACT

A surface and accessory system for temperature regulation and microclimate management of a patient includes a surface assembly including an outer covering and a spacer material. A pneumatic assembly is in fluid communication with the interior. The pneumatic assembly includes a housing. A blower is disposed within the housing. A flow rate valve is operably coupled to the discharge outlet of the blower. The flow rate valve is configured to adjust a discharge flow rate of the blower. A controller is communicatively coupled to the blower and the flow rate valve. The controller is configured to adjust at least one of an operating speed of the blower and a restriction setting of the flow rate valve to selectively generate a cooling effect and a heating effect with airflow directed by the blower through the surface assembly.

### CROSS-REFERENCE TO RELATED APPLICATION

This application claims priority to and the benefit under 35 U.S.C. § 119(e) of U.S. Provisional Application No. 63/591,519, filed on Oct. 19, 2023, entitled “HEATING AND MICROCLIMATE MANAGEMENT SURFACE SYSTEM FOR PATIENT SUPPORT APPARATUS,” the disclosure of which is hereby incorporated herein by reference in its entirety.

### FIELD OF THE DISCLOSURE

The present disclosure generally relates to a heating and microclimate management system, and more particularly to a temperature regulating surface and accessory system for a patient support apparatus that can use an airflow to generate both a heating effect and a cooling effect.

### SUMMARY OF THE DISCLOSURE

According to one aspect of the present disclosure, a temperature regulating and microclimate management surface system for a patient support apparatus includes a surface assembly including an outer covering having an inlet port and an outlet port and a spacer material disposed within an interior of the outer covering. A pneumatic assembly is in fluid communication with the inlet port. The pneumatic assembly includes a housing and a blower disposed within the housing. The blower is configured to direct air through the spacer material via the inlet port. A controller is communicatively coupled to the pneumatic assembly. The controller is configured to activate the blower at a first operating speed to direct the air through the surface assembly and generate a cooling effect. The controller is also configured to generate a heating effect through the surface assembly. To generate the heating effect, the controller is configured to adjust the blower to a higher second operating speed to generate heat in the housing and warm the air for the blower to intake heated air and direct the heated air through the surface assembly, restrict a discharge flow rate of the blower with a flow rate valve to generate the heat in the housing and cause the blower to direct the heated air through the surface assembly, or recirculate exiting heated air from the outlet port of the surface assembly to the pneumatic assembly to be redirected through the surface assembly by the blower.

According to another aspect of the present disclosure, a heating and cooling surface system includes a surface assembly including an outer covering and a spacer material disposed within an interior of the outer covering. A pneumatic assembly is in fluid communication with the interior. The pneumatic assembly includes a housing. A blower is disposed within the housing, and the blower has an intake and a discharge outlet. A flow rate valve is operably coupled to the discharge outlet of the blower. The flow rate valve is configured to adjust a discharge flow rate of the blower. A controller is communicatively coupled to the blower and the flow rate valve. The controller is configured to adjust the flow rate valve to a first restriction setting for a cooling effect with airflow directed through the surface assembly by the blower and adjust the flow rate valve to a second restriction setting for a heated airflow to be directed through the surface assembly by the blower for a heating effect. The second restriction setting is higher than the first restriction setting to cause a greater restriction on the discharge flow rate from the blower to generate heat that is captured by the blower and produce the heating effect with the heated airflow.

According to another aspect of the present disclosure, a heating and cooling surface system includes a surface assembly including an outer covering and a spacer material disposed within an interior of the outer covering. A pneumatic assembly is in fluid communication with the interior. The pneumatic assembly includes an enclosure operably coupled to the interior of the outer covering via inlet tubing and recirculation tubing. A blower is disposed within the enclosure, and the blower is configured to direct an airflow through the spacer material. A switch valve is operably coupled to the recirculation tubing and the interior of the enclosure. A controller is communicatively coupled to the blower and the switch valve. The controller is configured to activate the blower at a first operating speed to generate a cooling effect with the airflow through the spacer material for a cooling mode, activate the blower at a second operating speed to generate a heating effect with the airflow through the spacer material for a heating mode, the second operating speed being greater than the first operating speed to generate heat captured within the airflow, and adjust the switch valve to a closed state in the heating mode to redirect the airflow from the surface assembly to the blower.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a side perspective view of a heating and microclimate management system that includes a surface assembly in fluid communication with a pneumatic assembly for providing heating and microclimate management, according to the present disclosure;

FIG. 2A is a schematic diagram of a heating and microclimate management system that includes a surface assembly in fluid communication with a pneumatic assembly illustrating an airflow path for providing heating and microclimate management, according to the present disclosure;

FIG. 2B is a schematic diagram of a heating and microclimate management system that includes a blanket in fluid communication with a pneumatic assembly illustrating an airflow path for providing heating and microclimate management, according to the present disclosure;

FIG. 2C is a schematic diagram of a heating and microclimate management system that includes a surface assembly and a blanket in fluid communication with a pneumatic assembly illustrating airflow paths for providing heating and microclimate management, according to the present disclosure;

FIG. 3 is a partially exploded side perspective view of a pneumatic assembly for a heating and microclimate management system, according to the present disclosure;

FIG. 4 is a side perspective view of a pneumatic assembly for a heating and microclimate management system with an upper shell of an enclosure and an upper portion of a housing omitted and with interior tubing illustrated in phantom below a circuit board, according to the present disclosure;

FIG. 5 is a partial side perspective view of a pneumatic assembly with a blower, according to the present disclosure;

FIG. 6 is a cross-sectional view of the pneumatic assembly of FIG. 5, taken along lines VI-VI, according to the present disclosure;

FIG. 7 is a graph relating to performance of a blower, illustrating flow versus pressure and flow versus change in discharge temperature relative to ambient temperature, according to the present disclosure;

FIG. 8 is a schematic diagram of a heating and microclimate management system that includes a pneumatic assembly and a surface assembly with a coverlet and a mattress, according to the present disclosure;

FIG. 9 is a schematic diagram of a heating and microclimate management system that includes a pneumatic assembly and a surface assembly with a microclimate management layer within a mattress, according to the present disclosure;

FIG. 10 is a schematic diagram of a heating and microclimate management system that includes a pneumatic assembly with a heater and a surface assembly with a coverlet and a mattress, according to the present disclosure;

FIG. 11 is a schematic diagram of a heating and microclimate management system that includes a surface assembly and a pneumatic assembly with a switch valve to recirculate air in a heating mode and exhaust air in a cooling mode, according to the present disclosure;

FIG. 12 is a schematic diagram of a heating and microclimate management system that includes a surface assembly and a pneumatic assembly with a heater and a switch valve to recirculate air in a heating mode and exhaust air in a cooling mode, according to the present disclosure;

FIG. 13 is a block diagram of a heating and microclimate management system, according to the present disclosure;

FIG. 14 is a side perspective view of a support apparatus with a heating and microclimate management system that includes a pneumatic assembly, a surface assembly, and a blanket for providing cooling and heating with an airflow, according to the present disclosure;

FIG. 15 is a flow diagram of a method of controlling a heating and microclimate management system to provide a heating effect and a cooling effect, according to the present disclosure; and

FIG. 16 is a flow diagram of a method of controlling a heating and microclimate management system to provide a heating effect with recirculation and a cooling effect, according to the present disclosure.

### DETAILED DESCRIPTION

The present illustrated embodiments reside primarily in combinations of method steps and apparatus components related to a heating and microclimate management system for a patient support apparatus. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

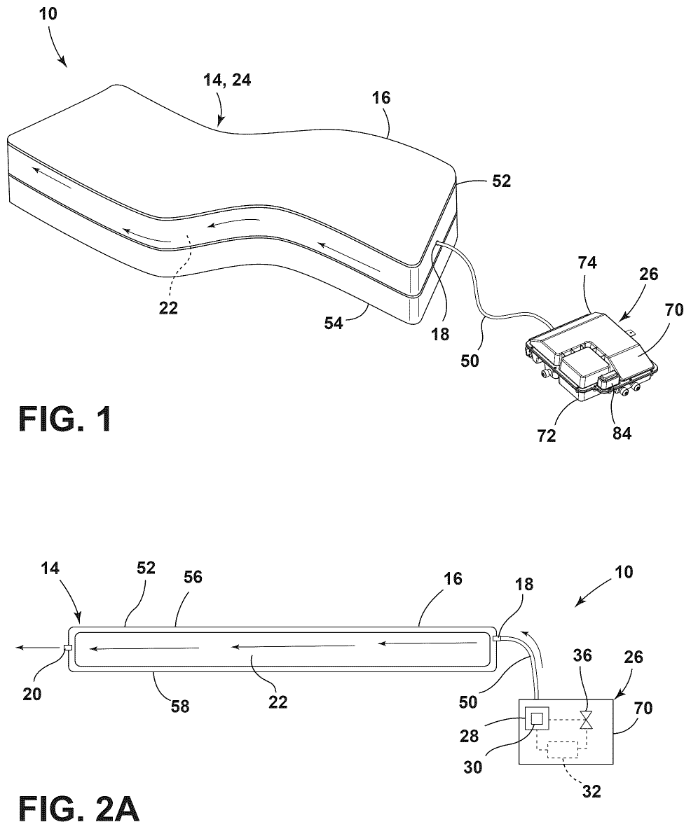

For purposes of description herein, the terms “upper,” “lower,” “right,” “left,” “rear,” “front,” “vertical,” “horizontal,” and derivatives thereof, shall relate to the disclosure as oriented in FIG. 1. Unless stated otherwise, the term “front” shall refer to a surface closest to an intended viewer, and the term “rear” shall refer to a surface furthest from the intended viewer. However, it is to be understood that the disclosure may assume various alternative orientations, except where expressly specified to the contrary. It is also to be understood that the specific structures and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

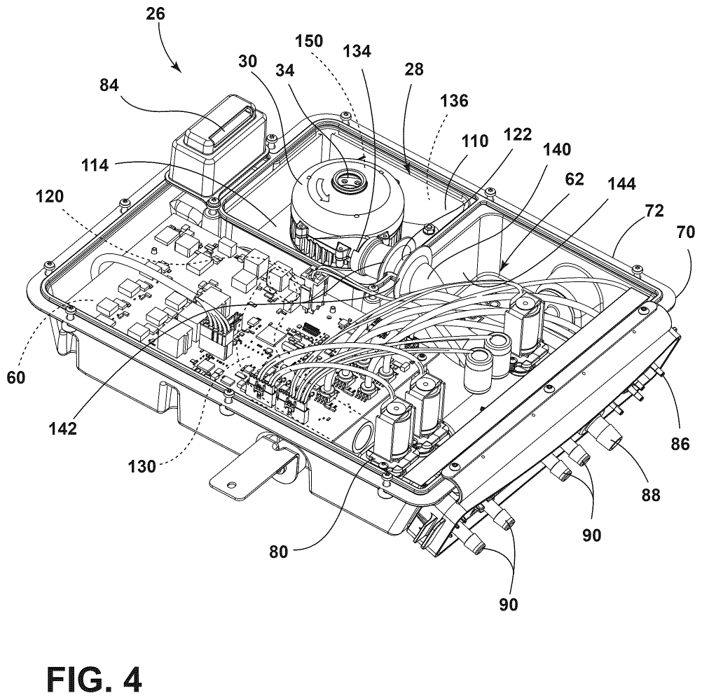

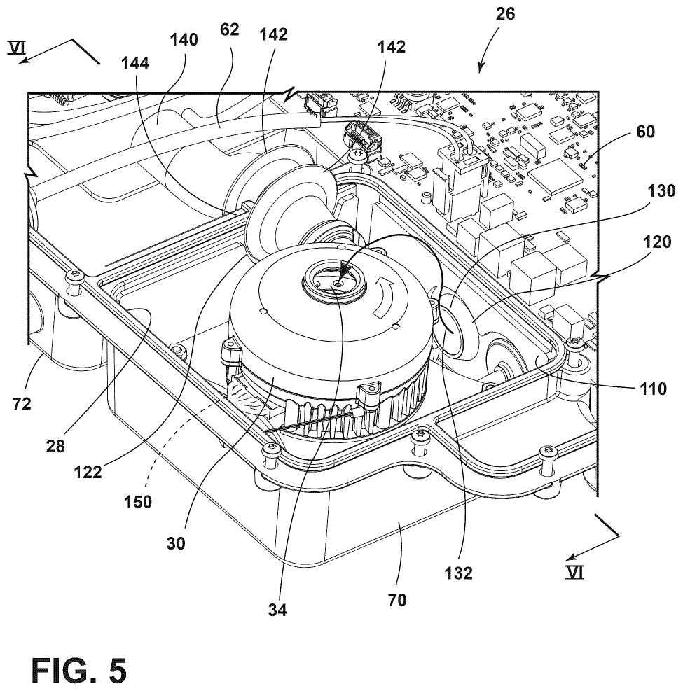

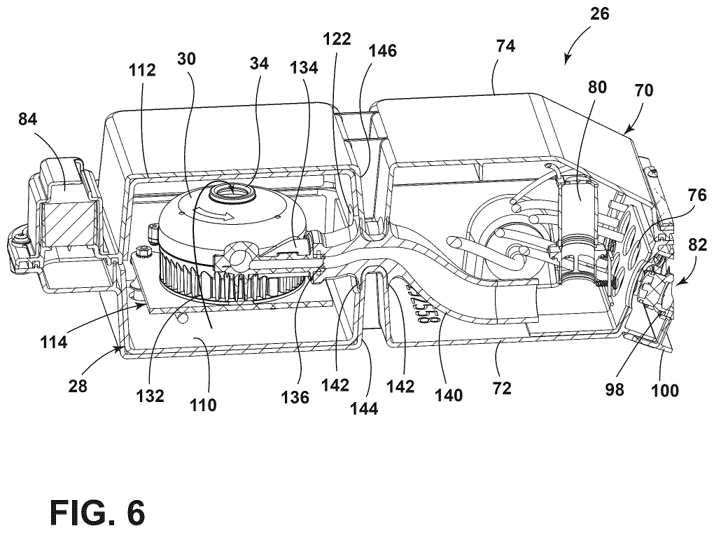

With reference to FIGS. 1-16, reference numeral 10 generally designates a temperature regulating or heating and microclimate management (MCM) system for a patient support apparatus 12, which may also be referred to as a temperature-regulating surface and accessory system 10 or a heating and cooling system 10. In certain aspects, the system 10 includes a surface assembly 14 for providing this combined functionality, where the surface assembly 14 includes an outer covering 16 having an inlet port 18 and an outlet port 20. A spacer material 22 is disposed within an interior of the outer covering 16. The surface assembly 14 may be an example of one or more temperature regulating/MCM components 24 included in the system 10.

A pneumatic assembly 26 is in fluid communication with the inlet port 18 of the surface assembly 14. The pneumatic assembly 26 includes a housing 28 operably coupled to the interior of the outer covering 16 via the inlet port 18 and a blower 30 disposed within the housing 28. The blower 30 is configured to direct air through the spacer material 22 via the inlet port 18. A controller 32 is communicatively coupled to the pneumatic assembly 26. The controller 32 is configured to activate the blower 30 at a first operating speed to generate a cooling effect with the airflow through the spacer material 22. The controller 32 is also configured to generate a heating effect by at least one of adjusting the blower 30 to a second operating speed, restricting a discharge flow rate from the blower 30, and recirculating the airflow from the outlet port 20 to the pneumatic assembly 26.

In examples where the controller 32 is configured to generate the heating effect by adjusting the blower 30 to the second operating speed, which is higher or greater than the first operating speed, the blower 30 is configured to generate heat in the housing 28 to produce heated air. The blower 30 is configured to draw the heated air through an intake 34 and direct the heated air through the spacer material 22 to, consequently, generate the heating effect with the airflow.

In examples where the controller 32 is configured to generate the heating effect by restricting the discharge flow rate, the controller 32 is communicatively coupled with a flow rate valve 36. The controller 32 is configured to adjust the flow rate valve 36 to restrict the discharge flow rate of the discharge airflow from the blower 30 to generate the heat in the housing 28 and produce the heated air. The blower 30 is configured to direct the heated air through the spacer material 22 to generate the heating effect with the airflow.

In examples where the controller 32 is configured to generate the heating effect by recirculating the airflow, the controller 32 is communicatively coupled with a switch valve 38. The controller 32 is configured to adjust the switch valve 38 to a closed state. The heated airflow exiting the interior of the outer covering 16 via the outlet port 20 (i.e., exiting heated air) is redirected and recirculated to the pneumatic assembly 26 to increase the heat in the airflow and generate the heating effect.

Additionally or alternatively, the system 10 can include a patient accessory 40, such as the illustrated example of the temperature regulating pad or blanket 40 that can be used for treating the patient. The accessories 40 can be positioned over the patient, such as the blanket 40, be worn by the patient, or otherwise be positioned against the patient to provide both the heating and cooling effect. With the blanket 40 as a non-limiting example, the blanket 40 includes an inlet port 42 and an outlet port 44. The blanket 40 also includes an outer covering 46 and a spacer material 48 disposed within an interior of the outer covering 46. The spacer material 48 in the blanket 40 may be thinner and/or provide less cushion than the spacer material 22 in the surface assembly 14. In this way, the surface assembly 14 can be used to support the patient thereon, and the blanket 40 can rest on or wrap around the patient. This allows the system 10 to have temperature regulating and MCM functionality below the patient via the surface assembly 14 and/or over the patient with the blanket 40.

Similar to the surface assembly 14, with the blanket 40, the pneumatic assembly 26 is in fluid communication with the inlet port 42, and the blower 30 is configured to direct air through the spacer material 48 via the inlet port 42. The controller 32 is configured to activate the blower 30 at the first operating speed to generate the cooling effect with the airflow through the spacer material 48. The controller 32 is also configured to generate the heating effect by at least one of adjusting the blower 30 to the second operating speed, restricting the discharge flow rate from the blower 30, and recirculating the airflow from the outlet port 44 to the pneumatic assembly 26 as described herein.

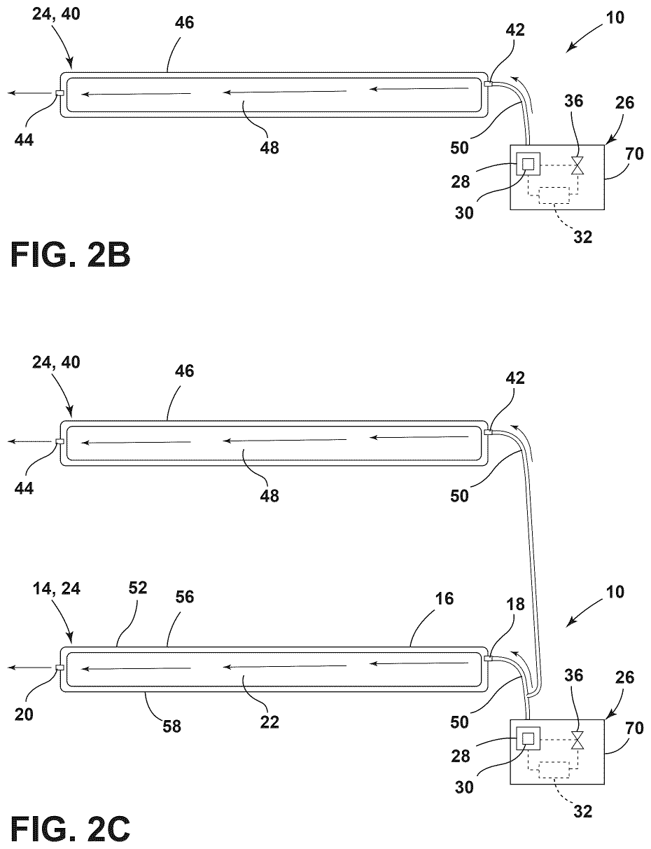

With reference to FIGS. 1-2C, the heating/MCM system 10 provides both heating and cooling temperature regulation for treating the patient. The heating/MCM system 10 includes the pneumatic assembly 26 and at least one temperature regulating/MCM component 24, such as the surface assembly 14 and/or the blanket 40, in fluid communication with one another via at least an inlet tubing 50. The pneumatic assembly 26 may be configured to couple to one of the surface assembly 14 and the blanket 40 or may be able to couple to both simultaneously via multiple inlet tubing 50, a branching inlet tubing 50 (e.g., forming diverging airflow paths to the surface assembly 14 and the blanket 40, see FIG. 2C), a coupling joint between inlet tubing 50, etc.

Referring to FIGS. 1 and 2A, the surface assembly 14 includes a topper or coverlet 52 and may also include a mattress 54. The coverlet 52 can be positioned on top of the mattress 54 or can be removably coupled to the mattress 54, such as with via buttons, snaps, hook-and-loop fasteners, ties, pins, zippers, etc. The coverlet 52 may also be integrated into the mattress 54 to form a single unit. In such examples, the coverlet 52 forms a layer, and generally a top layer, of the combined surface assembly 14. It is also contemplated that the surface assembly 14 may be the mattress 54 with a similar structure and function as the coverlet 52, with an increased thickness, and without a second support.

The coverlet 52 includes the outer covering 16 and the spacer material 22 within the outer covering 16. The outer covering 16 defines the inlet port 18 and the outlet port 20 at opposing ends of the outer covering 16. For example, the inlet port 18 may be defined in a foot end of the coverlet 52, while the outlet port 20 is defined in a head end of the coverlet 52. The outer covering 16 may be formed of an upper component 56 and a lower component 58 coupled together, such as through ultrasonic welding to form a fluid-tight seal. The inlet and outlet ports 18, 20 may be defined at a seam between the upper and lower components 56, 58. This configuration may be advantageous for coupling the inlet and outlet ports 18, 20 to the coverlet 52 in areas that may not affect the treatment or comfort of the patient supported on the coverlet 52. The opposing positions of the inlet and outlet ports 18, 20 allow the fluid (e.g., air) entering the interior of the coverlet 52 to pass through the interior and the spacer material 22 before exiting the coverlet 52 via the outlet port 20. Accordingly, the air flows through the spacer material 22 along a longitudinal extent of the coverlet 52 from one end to the opposing end.

The outer covering 16 can be moisture permeable and/or air impermeable. In a non-limiting example, the outer covering 16 may be constructed of nylon or a polyester material with a polyurethane coating that is moisture vapor permeable. The coverlet 52 may be considered a low air-loss coverlet 52, which broadly refers to a feature of the coverlet 52 that provides the airflow to assist in managing heat and humidity (e.g., microclimate) of the skin of the patient. Accordingly, the coverlet 52 may form the MCM system or layer for the temperature regulating system. As described further herein, air is directed through the coverlet 52 with the MCM function when the patient is positioned on the coverlet 52. Generally, the MCM function is utilized to provide the cooling effect for the patient.

Referring to FIG. 2B, the blanket 40, which is illustrated as the exemplary accessory 40, may have similar components with similar structure and/or functionality as the surface assembly 14. The blanket 40 includes the outer covering 46 and the spacer material 48 within the outer covering 46. The outer covering 46 may be formed via one or more components coupled together. The outer covering 46 defines the inlet port 42 and the outlet port 44. In a non-limiting example, the inlet port 42 and the outlet port 44 may be at opposing ends of the outer covering 46, such as the foot and head ends. The inlet port 42 and/or the outlet port 44 may also be defined at opposing side edges to allow airflow through the interior without airflow being expelled via the outlet port 44 proximate to a head area of the patient. The opposing positions of the inlet and outlet ports 42, 44 allow the air entering the interior of the blanket 40 to pass through the interior and the spacer material 48 before exiting the blanket 40 via the outlet port 44. The inlet port 42 and the outlet port 44 may be strategically placed based on use care for maximizing efficiency.

The outer covering 46 can be moisture permeable and/or air impermeable. Similar to the surface assembly 14, the blanket 40 may also be considered a low air-loss blanket 40, which broadly refers to a feature of the blanket 40 that provides the airflow to assist in managing microclimate of the skin of the patient. Air is directed through the blanket 40 with the MCM function, which is generally utilized to provide the cooling effect for the patient. Each component 24 included in the system 10 may have spacer material within an outer covering, along with at least one inlet port and at least one outlet port for directing air through the component 24 to provide the temperature regulating/MCM functionality or therapy to the patient.

The spacer material 22, 48 is generally constructed of materials having high fluid porosity and having some resistance against flattening. For the surface assembly 14, air can be directed through the spacer material 22, while the spacer material 22 also supports the weight of the patient on the coverlet 52 to reduce or prevent a bottoming effect felt by the patient. The spacer material 22, 48 is generally a three-dimensional network of fibers, knitted components, or mesh-like components that promotes airflow through the interior of the coverlet 52 or blanket 40, which can be constructed of various materials, such as, for example, textile fabrics, thermoplastic fibers, etc. In certain aspects, the spacer material 22, 48 may not include foam.

With reference still to FIGS. 1-2C, the heating/MCM system 10 includes the temperature regulating/MCM component 24, such as the coverlet 52 and/or the blanket 40, fluidly coupled with the pneumatic assembly 26 via at least the inlet tubing 50. In certain aspects, the mattress 54 may be separate from the coverlet 52 and include bladders for providing pressure redistribution or therapies to the patient. In such examples, the mattress 54 may also be in fluid communication with the pneumatic assembly 26. The pneumatic assembly 26 may be electrically and/or fluidly coupled with the surface assembly 14, the support apparatus 12 on which the surface assembly 14 is positioned, and/or the blanket 40. For example, various components of the pneumatic assembly 26 may be powered and controlled via electrical communication with the support apparatus 12. Additionally, the pneumatic assembly 26 includes a circuit board 60. Various electrical connectors 62 extend from the circuit board 60 to components within the pneumatic assembly 26, such as the blower 30, or extend out of the pneumatic assembly 26.

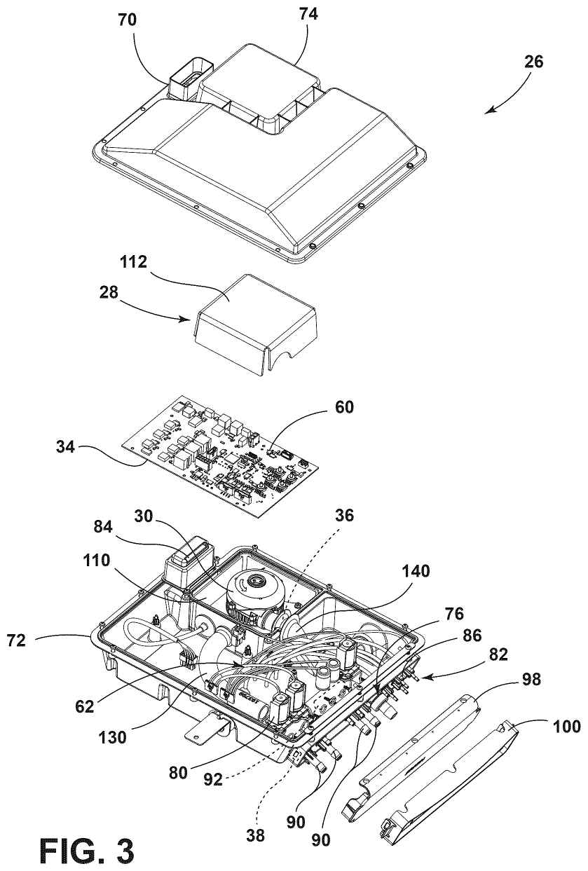

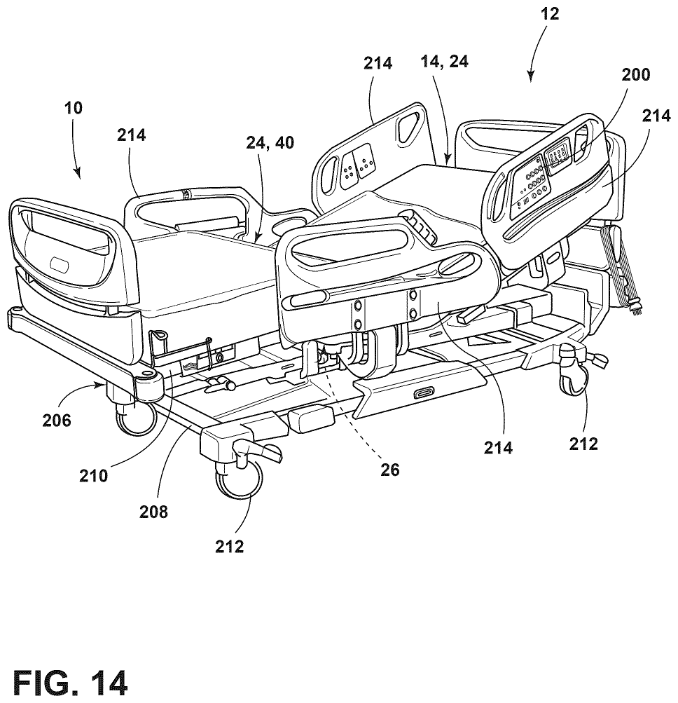

With reference to FIGS. 3-6, the pneumatic assembly 26 may be integrated into the support apparatus 12 (FIG. 14) or may be a separate assembly that can be selectively positioned on or supported by the support apparatus 12. The separate pneumatic assembly 26 may be advantageous for use with the blanket 40 (FIG. 2B) or other accessory 40 that can be more easily transported with the patient separate from the support apparatus 12. The pneumatic assembly 26 includes an enclosure 70, which includes a lower shell 72 for supporting the various components therein, and an upper shell 74 for enclosing the components within the interior of the enclosure 70. The enclosure 70 defines a plurality of openings 76 generally along one sidewall of the lower shell 72 to provide communication between the components within the enclosure 70 and other components outside the enclosure 70, such as the surface assembly 14.

The pneumatic assembly 26 includes an interface connector assembly 80 with various interface connectors 82 that extend through the openings 76 in the enclosure 70. The interface connectors 82 support various features for connection to the surface assembly 14 and/or blanket 40. The interface connectors 82 are illustrated on an opposing side of the pneumatic assembly 26 compared to an inlet projection 84. However, the interface connectors 82 can be in any practicable location to utilize or maximize the efficiency of the pneumatic assembly 26. The interface connectors 82 can include sense ports or fittings 86, at least one system coupling 88 (e.g., for coupling with the surface assembly 14 and/or the blanket 40), and bladder ports or hose fittings 90, which couple to the interface connectors 82 and external tubing or hoses. In this way, air can be drawn into the pneumatic assembly 26 via the inlet projection 84 on one side of the enclosure 70 and air can be directed out of the pneumatic assembly 26 via the system coupling(s) 88 and the hose fittings 90 on an opposing side of the enclosure 70. This may allow a closer relationship between the inlet projection 84 and the external environment, as well as a closer relationship between the interface connectors 82 and the surface assembly 14. The various connections to the pneumatic assembly 26 with the interface connectors 82 are outside of the enclosure 70.

The system coupling 88 and the hose fittings 90 may be in fluid communication with components inside and outside the enclosure 70, as well as one another. Further, the system coupling 88 and the hose fittings 90 may be operable between opened and closed states via valves 92 that may be operably coupled with the interface connector assembly 80. Accordingly, a single blower 30 may be utilized for the various functions of the system coupling 88 and the hose fittings 90 by opening and closing the valves 92 to direct the airflow.

The inlet projection 84 generally provides fluid communication between the blower 30 and an external area. For example, the inlet projection 84 allows the blower 30 to draw ambient air into the pneumatic assembly 26 to be directed by the blower 30 through the surface assembly 14 and/or the blanket 40. The inlet projection 84 may be disposed proximate to the housing 28 and in fluid communication with the interior of the enclosure 70. This configuration allows the blower to draw the ambient air through the inlet projection 84, through the interior of the enclosure 70, and into the housing 28. Moreover, the air being drawn through the interior of the enclosure 70 may assist with cooling other components within the pneumatic assembly 26.

The hose fittings 90 may be fill line fittings, which are configured to couple to hoses that direct fluid to and/or from various bladders in the mattress 54. These bladders may be, for example, turn bladders, a head bladder, and/or a seat bladder. An additional or alternative number of hose fittings 90 may be utilized based on the bladders in the heating/MCM system 10 or mattress 54. The sense fittings 86 may sense pressure in components of the heating/MCM system 10, such as in the bladders and/or the MCM system (e.g., within the coverlet 52 and/or blanket 40, see FIGS. 2A and 2B). The sense fittings 86 are generally communicatively coupled with the circuit board 60 to communicate the sensed pressure information to the controller 32. The controller 32 may use the sensed pressure information to adjust a treatment or therapy provided to the patient, provide additional comfort, alert a caregiver regarding a function of the heating/MCM system 10, etc.

Referring still to FIGS. 3-6, the system coupling 88 is disposed between the hose fittings 90 and the sense fittings 86. The system coupling 88 may be larger than the hose fittings 90 and sense fittings 86, which can be advantageous for directing more air through the system coupling 88 to the surface assembly 14. Moreover, generally, an increase in airflow is beneficial for providing the heating or cooling effect across the surface assembly 14 and/or blanket 40. The system coupling 88 is configured to couple with the connecting inlet tubing 50, which fluidly couples the pneumatic assembly 26 with the interior of surface assembly 14 and/or the interior of the blanket 40. Additional system couplings 88 may be included based on the number of temperature regulating/MCM components 24 are in the system 10. Valves or valve packs may be utilized to direct air through the system couplings 88 based on the components 24 and/or mode of the system 10.

Each of the hose fittings 90, the sense fittings 86, and the system coupling(s) 88 may include a seal, such as an O-ring, to provide an airtight seal to the pneumatic assembly 26. The hose fittings 90, the sense fittings 86, and the system coupling 88 also extend through support plates 98, 100, which extend along the sidewall of the enclosure 70 and couple to the enclosure 70.

The housing 28 is included within the enclosure 70 and has a lower portion 110 and an upper portion 112. The housing 28 generally defines a cavity for the blower 30 of the pneumatic assembly 26. This configuration provides a more direct airflow path from the inlet projection 84 to the blower 30 and from the blower 30 to the system coupling 88 while reducing airflow within the remainder of the enclosure 70. The housing 28 also assists with retaining heat proximate to the blower 30 to generate the heating effect as described herein.

The blower 30 is supported on a support plate 114, which assists with coupling the blower 30 to the housing 28, as well as directing the electrical connector 62 between the blower 30 and the circuit board 60. The housing 28 defines an inlet opening 120 and an outlet opening 122. The inlet opening 120 is defined in the lower portion 110, and the outlet opening 122 is defined between the lower and upper portions 110, 112. In this way, the lower and upper portions 110, 112 each define a groove, which align to mate with one another and form the outlet opening 122.

Referring still to FIGS. 3-6, an interior inlet tubing 130 extends from proximate to the interface connector assembly 80 to the inlet opening 120 within the enclosure 70. The interior inlet tubing 130 is in fluid communication with the inlet projection 84 and, consequently, an area external to the pneumatic assembly 26. The interior inlet tubing 130 generally has an aperture or opening, illustrated on a side of the inlet tubing 130 proximate to the interface connector assembly 80 for the blower 30 to draw air into the interior inlet tubing 130. The interior inlet tubing 130 extends below the circuit board 60 and to the interior inlet opening 120 of the housing 28. In this way, the interior inlet tubing 130 provides fluid communication between the cavity in the housing 28 and the inlet projection 84 for the blower 30 to draw ambient air into the housing 28. Further, the air can be drawn through the inlet projection 84, through the interior of the enclosure 70, into the interior inlet tubing 130, and then into the housing 28.

Generally, the inlet opening 120 is defined proximate to a base of the blower 30 and adjacent to the support plate 114. An intake 34 for the blower 30 is at a top of the blower 30 at an opposing end relative to where the blower 30 is supported on the support plate 114. Accordingly, the inlet opening 120 is proximate to a bottom of the housing 28, while the intake 34 of the blower 30 is proximate to a top of the housing 28. This configuration allows the blower 30 to draw air through the interior inlet tubing 130, along a height of the blower 30 and a height of the housing 28, as indicated by arrow 132, and into the top intake 34. This flow path along the height of the blower 30 and the housing 28 allows the air to be heated and/or capture heat generated by the blower 30 before being drawn into the intake 34 for the heating effect, as described herein.

The blower 30 includes a nozzle 134 defining a discharge outlet 136. The nozzle 134 extends from the side of the blower 30 and is aligned with the outlet opening 122 of the housing 28. An interior outlet tubing 140 is coupled to the nozzle 134 over the discharge outlet 136. The interior outlet tubing 140 extends from the cavity of the housing 28, through the outlet opening 122, and to the interface connector assembly 80. The interior outlet tubing 140 includes a radial stopper 142, or multiple radial stoppers 142, that abut or engage the housing 28 to assist in retaining the relationship between the housing 28, the blower 30, and the interior outlet tubing 140. The radial stoppers 142 are arranged on opposing sides of interior walls 144, 146 of the enclosure 70, which extend from or are formed by the lower shell 72 and the upper shell 74, respectively. The interior walls 144, 146 assist with positioning the housing 28 in the enclosure 70. The radial stoppers 142 engage the interior wall 144 to maintain the position of the interior outlet tubing 140, including during operation and adjustment of the blower 30.

Referring still to FIGS. 3-6, the blower 30 is configured to direct air through the discharge outlet 136, through the interior outlet tubing 140, and through the system coupling 88. The connecting inlet tubing 50 couples to the system coupling 88 outside of the enclosure 70 to receive the air from the blower 30 and direct the air to, and consequently through, the surface assembly 14 and/or blanket 40. In certain aspects, the pneumatic assembly 26 may include multiple system couplings 88 for engaging multiple inlet tubing 50 to direct the air to various components 24 of the system 10, including the surface assembly 14, the blanket 40, and/or other accessories 40.

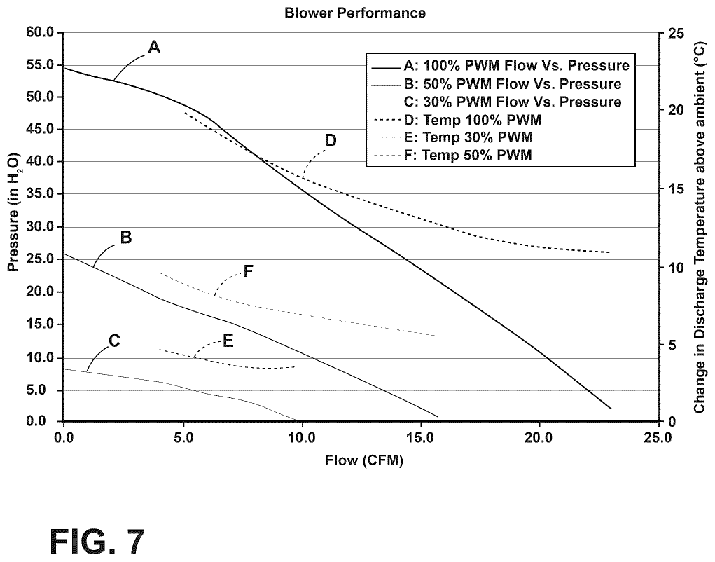

Referring still to FIGS. 3-6, as well as FIG. 7, in various examples, the pneumatic assembly 26 may also include the flow rate valve 36 or multiple flow rate valves 36 in fluid communication with the discharge air from the blower 30. The pneumatic assembly 26 may include a valve pack 36 with a combination of multiple valves 36 or with a single variable valve 36 for adjusting the discharge flow rate of the discharge air from the blower 30. In certain aspects, the flow rate valve 36 may be operably coupled to the blower 30 and/or the interior outlet tubing 140. The flow rate valve 36 is configured to selectively restrict the discharge airflow or discharge airflow rate generated by the blower 30. For example, the flow rate valve 36 is operable between multiple restriction settings, which can range from no or minimal restriction (e.g., about 0% restriction), increasing the discharge airflow rate, and significant or full restriction (e.g., up to about 100% restriction), decreasing the discharge airflow rate. It is also contemplated that one or more flow rate valves 36 may be utilized at the inlet projection 84 of the pneumatic assembly 26 or the intake 34 of the blower 30 in combination with or in lieu of the flow rate valve 36 at the discharge outlet 136 to restrict the airflow and generate heat.

In low restriction settings, the flow rate valve 36 allows for the discharge air to be directed from the discharge outlet 136 and through the interior outlet tubing 140 with minimal or no impingement to add minimal or no heat to the discharge air. Accordingly, the increased flow rate reduces pressure in the pneumatic assembly 26 and reduces the generation of heat by the blower 30. This condition is advantageous for generating the cooling effect. The low restriction settings may restrict the airflow to less than about 50%, less than about 25%, less than about 15%, less than about 10%, or less than about 5%. In non-limiting examples, the low restriction settings may be less than about 15% restriction compared to a maximum allowable airflow (e.g., at about 0% restriction or without the flow rate valve 36).

In high restriction settings, the size of space through which the discharge airflow is directed is reduced, which increases pressure upstream of the restriction. This increase in pressure causes the air temperature to increase proximate to the blower 30 as more electrical energy is converted to heat since the proportionate mechanical output is lower (e.g., relatively low airflow and high pressure). The high restriction settings may restrict the airflow greater than about 50%, greater than about 75%, greater than about 85%, greater than about 90%, or greater than about 95%. In non-limiting examples, the high restriction settings may be greater than about 75% restriction compared to the maximum allowable airflow. This condition is advantageous to heat the air directed by the blower 30 and provide the heating effect. The amount of restriction may also depend on the amount of heat to be generated and the temperature of the discharge air to be generated as described herein.

With reference still to FIGS. 3-7, the heating/MCM system 10 is configured to provide both the heating effect and the cooling effect with the airflow directed through the surface assembly 14. The heating/MCM system 10 can selectively switch between the cooling effect and the heating effect based on the control of the blower 30 and/or the flow rate valve 36. It is contemplated that the heating and cooling effects can be generated with the blower 30 independently, which may be advantageous when the flow rate valve 36 is omitted from the heating/MCM system 10, the flow rate valve 36 independently, or both the blower 30 and the flow rate valve 36.

One or more of the speed of the blower 30, the duty cycle of the blower 30, and restriction by the flow rate valve 36 may be adjusted to produce a selected temperature effect for the patient. A change in the flow speed (e.g., the speed of the blower 30) or the restriction at the discharge outlet 136 can change the heat output that can be used to heat or cool the skin of the patient. In certain aspects, the input power supply conditions for the blower 30 can be changed to adjust the duty cycle and/or operate the blower 30 at different speeds. The blower 30 includes an integrated electrical motor 150, which can be controlled by pulse width modulation (PWM) to change the operating speed of the blower 30.

At each different operating speed, the blower 30 utilizes a different amount of electrical energy. Out of the energy the blower 30 is utilizing, part of the electrical energy is radiated as heat. In conventional systems, this generation of heat is generally considered an inefficiency. In such conventional systems, the heat is often dissipated away. In the heating/MCM system 10 disclosed herein, this heat is generated and controlled to be used to warm the patient. The remaining electrical energy that is not radiated as heat is converted into mechanical output and contributes to the airflow and pressure. In this way, the heating/MCM system 10 can leverage the discharge air temperature of the blower 30 as it relates to the PWM at which the blower 30 is operated. Accordingly, by changing the speed of the electrical motor-equipped blower 30 by providing more or less electrical energy and/or by adjusting the flow rate valve 36 to more or less restrictive settings, the heating/MCM system 10 can increase and decrease the heat output of the discharge air.

When the heating/MCM system 10 is operating with the blower 30 at a lower speed and with less restriction to the discharge air, the heating/MCM system 10 is operating in a cooling condition or mode as ambient air is utilized in the heating/MCM system 10 for cooling the patient, which provides the MCM function for the heating/MCM system 10. To switch to a heating condition or mode, the heating/MCM system 10 can adjust the blower 30 to a higher speed or higher duty cycle and/or adjust the restriction at the discharge outlet 136 of the blower 30.

For example, the blower 30 may be maintained at the same or a single operating speed for providing the heating and cooling effect, with a change in the restrictive setting generating the heat for the heating effect. In another non-limiting example, the speed of the blower 30 may be increased through PWM to generate additional heat with the restrictive setting of the flow rate valve 36 being maintained at a same setting between the heating and cooling effects. In such examples, more electrical energy is provided to the blower 30, resulting in an increase in energy that is radiated as heat. In a further non-limiting example, both the blower 30 and the flow rate valve 36 are adjusted to generate or reduce the generation of heat.

In the example illustrated in FIG. 7, the graph illustrates a relationship between pressure (in “H20) and flow (CFM) and a relationship between a change in temperature relative to ambient (in ° C.) and flow (CFM). As illustrated, the change in temperature in the discharge air from the blower 30 is based on a change relative to ambient temperature, which is about 21° C. Accordingly, 5° C. on the graph in FIG. 7, indicates a change in temperature of about 5° C. relative to ambient temperature or about 26° C. In this way, a temperature change of 0° C. indicates that the discharge air is about the same temperature as ambient.

Lines A-C in the graph show the relationship between airflow and pressure at the different PWM settings of the same blower 30. For example, in Line A, when the blower 30 is operating at 100% PWM and when there is no or minimal airflow, the pressure is about 55 “H₂0. This is generally full restriction by the flow rate valve 36, which increases the pressure. As the airflow increases, the pressure decreases. Line B and Line C show a similar relationship. For Line B, when the blower 30 is operating at 50% PWM and there is minimal or no airflow, the pressure is slightly above 25 “H₂0, with pressure decreasing as airflow increases. Similarly, for Line C, when the blower 30 is operating at 30% PWM and there is minimal or no airflow, the pressure is slightly below 10 “H₂0, with pressure decreasing as airflow increases. Additionally, Lines A-C illustrate that an increase in PWM of the blower 30 causes an increase in pressure at similar or the same airflow rates. As described herein, the change in PWM can be generated by change in electrical energy provided to the electrical motor 150 associated with the blower 30 and the change in flow rate can be generated by changing the restrictive setting of the flow rate valve 36.

Additionally, lines D-F illustrate the temperature changes that can be generated based on the airflow (e.g., as changed with the flow rate valve 36). The lower the airflow, the higher the change in temperature of the discharge air relative to ambient. In the graph of FIG. 7, the “Temp 100% PWM” Line D illustrates an example of the temperature of the discharge air for the heating effect with the blower 30 operating at 100% PWM. At a greater restrictive setting by the flow rate valve 36 (e.g., less airflow), the pressure is about an airflow of about 5 CFM, the temperature is about 20° C. above ambient. When the airflow is at about 23 CFM, the temperature decreases to about 11° C. Accordingly, depending on the restriction by the flow rate valve 36, the discharge air can be between about 10° C. and about 20° C. above ambient temperature. With ambient temperature in many operating rooms being about 21° C., this can increase the discharge air through the surface assembly 14 or the blanket 40 to a temperature of about 41° C., which is higher than the average core body temperature of about 37° C. This heating effect can be a treatment-based heating effect to provide treatment for those with various conditions, such as hypothermia.

The “Temp 30% PWM” Line E illustrates an example of the temperature of the discharge air for the cooling effect with the same blower 30 as Line D operating at 30% PWM. Based on the restriction in the airflow, the temperature increases between about 4.5° C. and about 5° C. above ambient. This significantly lesser temperature increase relative to ambient allows the airflow through the surface assembly 14 to provide the cooling effect for the MCM function.

The heating/MCM system 10 can also be used to generate a comfort-based heating effect, where the heating/MCM system 10 provides air that is warmer than the MCM function but cooler than the treatment-based heating effect. For example, “Temp 50% PWM” Line F illustrates the temperature above ambient based on airflow for the blower 30 operating at 50% PWM. At an airflow of about 5 CFM, the discharge air is about 10° C. above ambient. At an airflow of about 15 CFM, the discharge air is about 5° C. above ambient. Accordingly, the air flowing through the surface assembly 14 can be between about 5° C. and about 10° C. above ambient to provide additional warmth to the patient for comfort but less than the amount of heat for treating the patient. Lines E-F illustrate that an increase in PWM of the blower 30 causes an increase in temperature at similar or the same airflow rates. Lines E-F also illustrate the combination of PWM and flow rate for generating heat and an amount of heat for the heating effect or for minimizing heat to generate the cooling effect.

The caregiver and/or the controller 32 can adjust the speed of the blower 30 via PWM, the duty cycle of the blower 30, and/or the flow rate valve 36 to adjust the generation of heat and, consequently, the temperature effect for the discharge air that flows through the surface assembly 14 that supports the patient and/or any component 24 (including the blanket 40) for treating the patient. These three factors can be adjusted independently or in any combination to provide a more customizable and adaptable heating/MCM system 10. Whether the blower 30, the flow rate valve 36, or both are adjusted may depend on the type of treatment provided to the patient, how quickly the heating/MCM system 10 may change between heating and cooling, the configuration of the blower 30, the performance of the blower 30, etc.

In this way, adjustment of the flow rate valve 36 and/or the blower 30 may be utilized to provide the cooling effect or the heating effect with the same heating/MCM system 10. This may be advantageous for providing additional therapies for the patient, such as skin cooling and moisture wicking with the MCM function, heating for conditions such as hypothermia, and/or cooling or heating for providing additional comfort to the patient. The air may be the coolest temperatures relative to ambient to provide the cooling MCM effect, may be slightly warmed relative to ambient to provide additional comfort for the patient with the heating effect, and the warmest relative to ambient to provide the heating effect for treating the patient.

For example, the cooling effect generally provides the MCM function through the surface assembly 14. The MCM function provides cooler air flowing through the spacer material 22 to provide the moisture wicking treatment. This configuration wicks away moisture from the skin of the patient by blowing air beneath the patient, which is advantageous for preventing skin conditions that may be caused by lying on the surface assembly 14 for an extended period of time. This may be advantageous for reducing or preventing pressure injuries which can be caused by remaining positioned on surface assembly 14 for an extended period of time. In certain aspects, the patient may be subject to moisture wicking treatment and may have certain conditions that may result in reduced body temperature, such as hypothermia. The heating/MCM system 10 can switch between moisture wicking treatment and hypothermia treatment. The temperature of the air flowing through the spacer material 22 is adjusted to provide the selected temperature effect for the patient or the treatment.

The blanket 40 may also be utilized for the MCM cooling function to wick away moisture from the skin of the patient by blowing air over the patient. Additionally or alternatively, in configurations utilizing both the surface assembly 14 and the blanket 40 concurrently, one of the surface assembly 14 and the blanket 40 can provide the cooling effect while the other of the surface assembly 14 and the blanket 40 can provide the heating effect. This may provide multiple therapies to the patient at once. For example, this may be advantageous for patients who may be more likely to develop pressure injuries but are also being treated for hypothermia. It is also contemplated that the heating/MCM system 10 can alternate between moisture wicking treatment and hypothermia treatment in the surface assembly 14 and the blanket 40 (e.g., the surface assembly 14 providing cooling and the blanket 40 providing heating and switching to the surface assembly 14 providing heating and the blanket 40 providing cooling).

In examples with two different temperatures being used for the surface assembly 14 and the blanket 40, separate pneumatic assemblies 26 can be used. Alternatively, separate system couplings 88 may be used with valves for directing airflow, where the airflow may adjust based on the setting of the pneumatic assembly 26 (i.e., heating or cooling). For example, when the pneumatic assembly 26 is generating the heating effect, the valve(s) may be set to direct air to the blanket 40, and when the pneumatic assembly 26 is generating the cooling effect, the valve(s) may be set to direct air to the surface assembly 14. One component 24 or multiple components 24 may be included in the system 10 depending on the therapies to be provided to the patient, the diagnosis of the patient, etc.

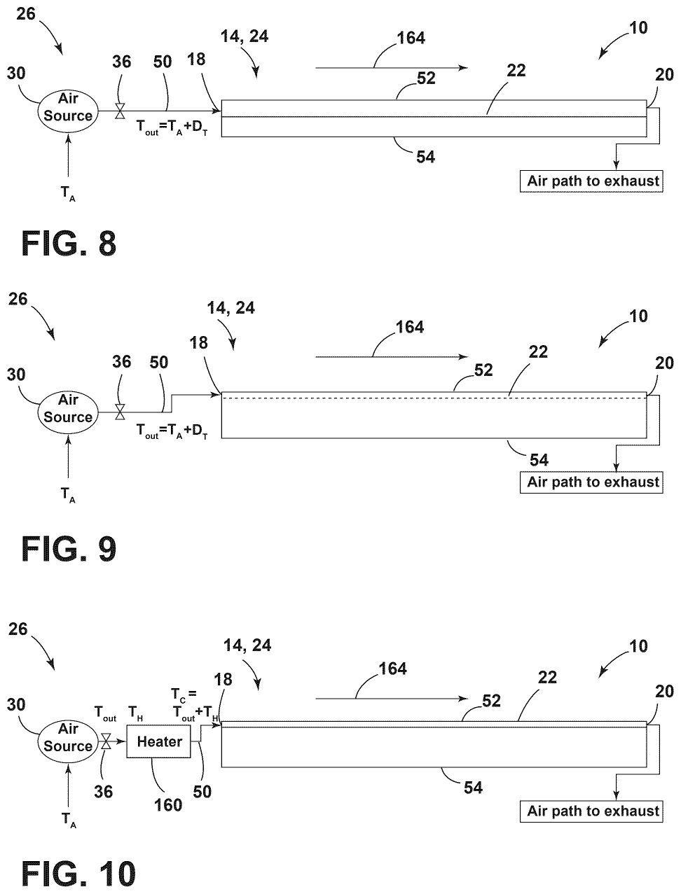

Referring to FIGS. 8-10, in the illustrated non-limiting examples, the surface assembly 14 may have a variety of configurations for providing the MCM cooling effect and the heating effect. For example, as illustrated in FIG. 8, the surface assembly 14 includes the coverlet 52 and the mattress 54 as separate components. The pneumatic assembly 26 directs the airflow through the coverlet 52 to produce the heating and cooling effect. Accordingly, the inlet port 18 and the outlet port 20 for the temperature effect airflow are coupled to the outer covering 16. The air from the blower 30 is directed through the inlet port 18, through the spacer material 22, and is exhausted through the outlet port 20. The airflow for the heating and cooling effect may not be directed through the mattress 54.

In an additional or alternative configuration, as illustrated in FIG. 9, the surface assembly 14 may include the coverlet 52 forming an MCM layer within the mattress 54. Accordingly, in this configuration, the MCM layer is integrated into the mattress 54, forming a separate layer divided from the remainder of the mattress 54 by the outer covering 16 that extends around the spacer material 22. The airflow is directed through the spacer material 22 within the MCM layer and is not or not significantly directed through the remainder of the mattress 54.

In another additional or alternative configuration, the surface assembly 14 is an MCM mattress 54, which includes the outer covering 16 around the spacer material 22. This MCM mattress 54 may not include other components within the interior of the mattress 54. The MCM mattress 54 may have a thicker spacer material 22 or a layer of spacer material 22 over a layer of foam to provide the path for the airflow and support for the patient. This configuration may or may not be used with an additional support structure.

In additional non-limiting examples, the surface assembly 14 may be configured as the mattress 54 without including the spacer material 22. In such examples, the surface assembly 14 may include additional structures or features within the interior of the surface assembly 14. Further, the inlet port 18 and the outlet port 20 for the temperature effect airflow are coupled to the outer covering 16 and in fluid communication with one another via the interior of the surface assembly 14. The air from the blower 30 is directed through the inlet port 18, through the interior of the surface assembly 14, and is exhausted through the outlet port 20. The airflow for the heating and cooling effect may be routed through a portion or the entire interior of the surface assembly 14. The accessories 40 and other components 24 may have a single layer or multiple separate layers similar to the configurations described with respect to the surface assembly 14, such as those described in relation to FIGS. 8-10.

Referring now to FIGS. 8-12, the features for generating the heating effect with of the heating/MCM system 10 are illustrated in conjunction with the surface assembly 14. However, it is contemplated that the system 10 may include the same or similar components for generating the heating effect with the blanket 40 and other temperature regulating/MCM components 24 without departing from the teachings herein. In the illustrated examples, the airflow path for each configuration of the surface assembly 14 is through the spacer material 22 is at an upper or top of the surface assembly 14 for providing the temperature effect to the patient. The airflow in the surface assembly 14 close to the patient (e.g., separated by the outer covering 16) allows the airflow to cool or warm the skin of the patient. Ambient air is drawn into the pneumatic enclosure 70 by the blower 30 via the inlet projection 84. The air is drawn into the blower 30 and directed through the interior outlet tubing 140 and through the connecting inlet tubing 50 between the pneumatic assembly 26 and the surface assembly 14. The air is then directed through the inlet port 18 at one end of the surface assembly 14, flows through the spacer material 22 along the longitudinal extent of the surface assembly 14, and then through the outlet port 20 to be exhausted into the environment.

When operating in the cooling mode to provide the MCM cooling effect, the blower 30 and/or the flow rate valve 36 are set for the blower 30 to generate less heat. This results in the air flowing through the surface assembly 14 being about or slightly warmer than ambient temperature. When operating in a first or comfort heating mode to provide the comfort-based heating effect, the blower 30 and/or the flow rate valve 36 are set for the blower 30 to generate more heat than the cooling effect but less than the treatment-based heating effect. The heat is captured or the air is warmed as the air flows in the housing 28, along the height of the blower 30. This path for the airflow proximate to the blower 30 that is radiating heat, allows the air to capture heat to be used for warming the patient.

When operating in a second or treatment heating mode to provide the treatment-based heating effect, the blower 30 and/or the flow rate valve 36 are set for the blower 30 to generate the most heat. The configuration of the housing 28 assists in retaining the heat proximate to the blower 30, reducing dissipation or exhaustion of heat from the enclosure 70. In this way, where conventional systems have inefficiencies due to the generation of heat that is to be dissipated away from the blower, the heating/MCM system 10 disclosed herein purposefully generates and controls the generation of additional heat to be captured by the blower 30 to direct warm air through the surface assembly 14.

With reference to FIG. 10, the heating/MCM system 10 may also include a heater 160. The heater 160 may be included in the pneumatic assembly 26 and/or the connecting inlet tubing 50. The heater 160 is configured to heat the discharge air being directed to the surface assembly 14. The heater 160 may be a cartridge or resistive heater. Generally, the air is directed across the heater 160 to be warmed by heat being radiated from the heater 160. Accordingly, the heater 160 may be in the flow path of the discharge air from the blower 30 to add additional heat to the discharge air. In various aspects or for certain periods of time, the heater 160 may be the primary source of heat for the heating effect.

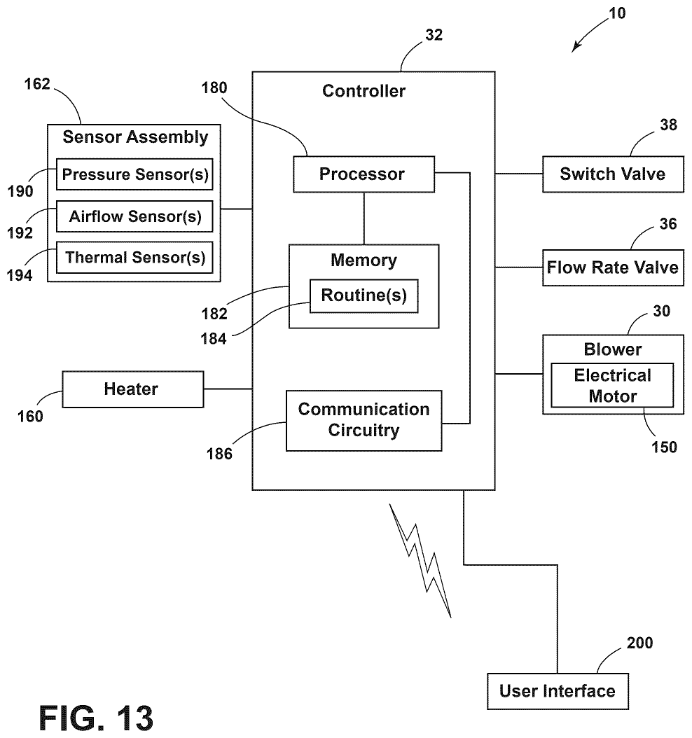

Additionally or alternatively, the heater 160 may be used in combination with the adjustment of the blower 30 and/or the flow rate valve 36. Accordingly, the heater 160 may be used to provide heat quickly while the blower 30 and the flow rate valve 36 generate heat. The heater 160 may remain activated when the heat is captured by the blower 30 or may be deactivated when the blower 30 is capturing sufficient heat based on the settings of the blower 30 and/or the flow rate valve 36. The heater 160 may also be deactivated when the discharge air reaches a predefined temperature, indicative of the blower 30 capturing sufficient heat. The heater 160 may also be advantageous for warming the patient with certain conditions, such as hypothermia, to further increase the temperature of the discharge air relative to ambient. The pneumatic assembly 26 may include a sensor assembly 162 (FIG. 13), which may assist in monitoring the temperature of the discharge air and/or the air in the housing 28 to control the heater 160 as described herein.

Referring again to FIGS. 8-10, in the illustrated examples, the temperature effect for the MCM cooling and the heating for comfort and treatment defines a one-way flow path, indicated by arrow 164, through the heating/MCM system 10. The blower 30 is configured to draw ambient air into the pneumatic assembly 26. The ambient air is at an ambient temperature T_{A}. In the MCM cooling mode or setting, the air is directed through the surface assembly 14 and exhausted from the outlet port 20 without a significant increase in temperature relative to T_{A}. This temperature may be Tₒᵤₜ, where Tₒᵤₜ=T_{A}+D_{T} and where D_{T} is any raise or increase in temperature due to the function of the blower 30.

In the heating mode or setting, the pneumatic assembly 26 is configured to generate and capture heat by adjusting the speed of the blower 30, adjusting the duty cycle of the blower 30, and/or adjusting the restrictive setting of the flow rate valve 36. The discharge air is warmed based on the adjustment to the blower 30 and/or the flow rate valve 36 to an outlet temperature Tₒᵤₜ. In examples with the heater 160, the heater 160 is configured to further warm the discharge air by a heater temperature T_{H} to a combined temperature T_{c}, which is Tₒᵤₜ+T_{H}. The warmed discharge air is directed through the surface assembly 14 and is exhausted via the outlet port 20.

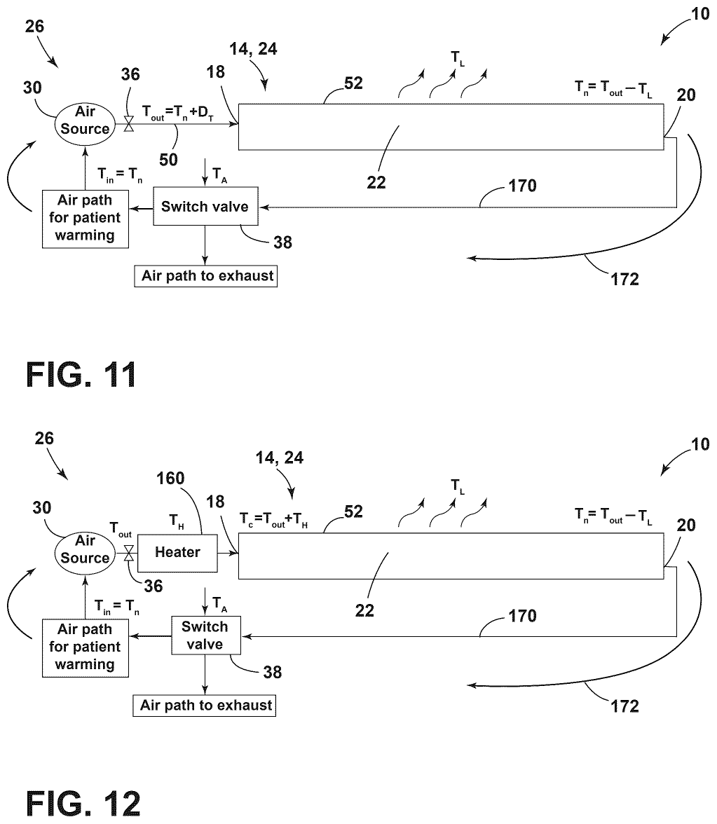

With reference to FIGS. 11 and 12, in additional or alternative examples, the air exiting the surface assembly 14 may be heated air that is re-used or re-cycled by the heating/MCM system 10 to maximize the heating effect and the efficiency of the heating/MCM system 10. In such aspects, the heating/MCM system 10 may be a closed loop system where the pneumatic assembly 26 is in fluid communication with surface assembly 14 via the connecting inlet tubing 50 and a recirculation tubing 170. In this way, the outlet port 20 of the surface assembly 14 is in fluid communication with the inlet projection 84 or other interface connectors 82 via the recirculation tubing 170.

The closed loop heating/MCM system 10 is substantially similar to the one-way heating/MCM system 10 for generating and capturing heat by adjusting the blower 30 and/or the flow rate valve 36. The closed loop heating/MCM system 10 can be used for the cooling effect, the comfort-based heating effect, and the treatment-based heating effect. Compared to the one-way heating/MCM system 10, the closed loop heating/MCM system 10 includes the recirculation tubing 170 and the switch valve 38 to redirect the exiting air from the surface assembly 14 back to the pneumatic assembly 26 in the heating mode, as indicated by arrow 172.

During the MCM operation for providing the cooling effect, the switch valve 38 is positioned for the blower 30 to draw ambient air into the heating/MCM system 10 through the switch valve 38. The ambient air at ambient temperature T_{A} is drawn into the pneumatic assembly 26 and through the blower 30. The discharge air is directed by the blower 30 from the pneumatic assembly 26 and through the surface assembly 14. The discharge air is at temperature Tₒᵤₜ, which is the ambient temperature T_{A} plus any temperature increase D_{T} due to the function of the blower 30 (such as illustrated with Line E in FIG. 7). The discharge air at temperature Tₒᵤₜ is directed through the surface assembly 14 and exits the surface assembly 14 through the outlet port 20 into the recirculation tubing 170. The recirculation tubing 170 directs the exiting air to the switch valve 38, which is in an exhaust or opened state to exhaust the air to the atmosphere. The opened state of the switch valve 38 allows the heating/MCM system 10 to continually draw in ambient air to provide the cooling effect to the patient.

Referring still to FIGS. 11 and 12, for generating the heating effect, the operation of the heating/MCM system 10 begins similar to the MCM cooling operation. The ambient air at ambient temperature T_{A} is drawn into the pneumatic assembly 26 via the blower 30 and the switch valve 38. The air is warmed to Tₒᵤₜ, which is the ambient temperature T_{A} plus any temperature increase D_{T} caused by operation of the blower 30 and/or the flow rate valve 36. The air at temperature Tₒᵤₜ is directed through the surface assembly 14. As the air travels through the surface assembly 14, the air may lose heat (i.e., heat lost to patient T_{L}) by transferring the heat to the skin of the patient. The air exiting the surface assembly 14 into the recirculation tubing 170 may be at temperature Tₙ, where Tₙ=Tₒᵤₜ−T_{L}.

The air is directed through the recirculation tubing 170 and toward the switch valve 38, which is in a recirculation or closed state. The switch valve 38 in the closed state redirects the air that has exited the surface assembly 14 back into the pneumatic assembly 26. The air at temperature Tₙ, which may also be referred to as an input temperature Tᵢₙ, is redirected into the pneumatic assembly 26. The air at Tₙ may be further warmed by the function of the blower 30 and/or the flow rate valve 36 to be the discharge temperature Tₒᵤₜ, where Tₒᵤₜ=Tᵢₙ+D_{T} and where D_{T} is the raise in temperature generated by the blower 30 and/or the flow rate valve 36. Tₙ is the temperature of the air in the nth cycle through the blower 30. Tₙ increases as “n” increases. The amount of increase in Tₙ may depend on heat loss to keep the skin of the patient warm.

Referring still to FIGS. 11 and 12, the ambient air is initially heated and directed through the surface assembly 14. The air generally loses some heat due to absorption by the patient. This exiting air, which is heated exiting air compared to ambient temperature, is recirculated to the blower 30. Accordingly, in this configuration, the warmer air (which is the heated air less the loss to the patient) is reused by the blower 30, allowing the heating/MCM system 10 to begin the cycle at the pneumatic assembly 26 with air that is above ambient, which is advantageous for warming the pre-warmed air to the predefined temperature more efficiently. The blower 30 and/or the flow rate valve 36 may generate the warmed air initially and then may utilize less energy, such as by restricting the airflow to a lesser amount or increasing the blower 30 to a lesser speed compared to an initial setting, and to warm the recycled air to a higher temperature.

For example, the ambient air, at 0° C. above ambient temperature, may be drawn into the blower 30 and increased in temperature to 5° C. above ambient temperature. The discharge air, at 5° C. above ambient, is directed through the surface assembly 14 where heat is lost by warming the patient skin. As an illustrative example, the heat loss may be about 2° C., resulting in the air exiting the surface assembly 14 to be at a temperature of 3° C. above ambient. This exiting air at 3° C. above ambient is then redirected back to the blower 30. Accordingly, for the second cycle through the heating/MCM system 10, the air drawn into the blower 30 is 3° C. higher than the first cycle when the air was at ambient temperature, resulting in a more efficient heating/MCM system 10 to increase this warmed air to the predefined temperature of 5° C. in this example. The heating/MCM system 10 may continue to recycle the air to provide the heating effect. Upon completion or termination of the heating function, the switch valve 38 can be adjusted to the exhaust state to exhaust the air into the atmosphere.

The degree of warming in the recirculating closed loop heating/MCM system 10 is less than the one-way heating/MCM system 10 after the initial warming, allowing the consumption of less energy by the heating/MCM system 10. The air exiting the surface assembly 14 is directed back through the blower 30 to again leverage the electrical energy to warm the air that is then directed or dispersed through the surface assembly 14.

Further, this heating/MCM system 10 is a closed loop, drawing in ambient air to begin the cycle and then recycling the same air. This provides a self-contained system that draws in ambient air initially and then recycles the air until the air is ultimately exhausted at the end of the heating treatment. The closed loop system reduces or prevents the introduction of contaminants into the heating/MCM system 10 and the air being directed through the spacer material 22. Moreover, the heating/MCM system 10 may be sealed after the initial intake of ambient air. Generally, the outer covering 16 is leak-proof or resistant to leaks. Accordingly, closing the airflow loop results in a contained and sealed system. The self-contained system with less, minimal, or no cross-contamination due to recirculation of the same air can reduce the risk of infection compared to conventional patient warming techniques.

Referring still to FIG. 12, in the recirculating configuration, the pneumatic assembly 26 may include the heater 160 to generate heat to be captured by the discharge airflow from the blower 30. In various aspects, the heater 160 may be the primary source of heat for the heating effect for a portion or an entirety of the time the heating effect is generated. Additionally or alternatively, the heater 160 may be utilized in combination with the heat generated by control of the blower 30 and/or the flow rate valve 36, similar to the one-way heating/MCM system 10 described herein.

In additional non-limiting examples, the heater 160 may be activated as the heating/MCM system 10 is drawing in the initial ambient air. The heater 160 may be used to generate heat for a predefined number of cycles through the heating/MCM system 10. After a predefined number of cycles or when the discharge air reaches a predefined temperature as sensed by the sensor assembly 162 (FIG. 13), the heater 160 may be deactivated. In such examples, the remainder of the cycles through the heating/MCM system 10 may utilize the control of the blower 30 and/or the flow rate valve 36 with the pre-warmed air for providing the heating effect. When utilizing the heater 160, the air being directed through the surface assembly 14 is at temperature Tₙ, where Tₙ=Tₒᵤₜ+T_{H}−T_{L}, where Tₒᵤₜ is the temperature of the discharge air from the blower 30, T_{H} is the temperature increase from the heater 160, and T_{L} is the loss of heat to the skin of the patient.

Referring again to FIGS. 8-12, the system 10 is illustrated with the surface assembly 14. It is contemplated that the effects can be generated in the blanket 40, other accessories 40, or any temperature regulating/MCM component 24 in the same or substantially similar manner, including in the one-way system 10 and/or the closed loop system 10. In this regard, the system 10 may be substantially similar to the configurations in FIGS. 8-12 with the blanket 40 or other component 24 in place of the surface assembly 14.

The system 10 described with respect to FIGS. 8-10 discloses a one-way airflow path and the system 10 described with respect to FIGS. 11 and 12 includes the closed loop configuration, recycling air that is directed through the surface assembly 14. The illustrated examples include the airflow through a single component 24, which is illustrated as the surface assembly 14. However, it is contemplated that the air may be directed through multiple components 24, such as through both the surface assembly 14 and the blanket 40 (or another system component 24) to provide the heating or cooling effect to the components of the system 10 in sequence before being exhausted via the final outlet or being directed through the recirculation tubing 170.

For example, the air may be directed from the pneumatic assembly 26 and to the surface assembly 14 through the inlet tubing 50. The air may be directed through the surface assembly 14 and through the outlet port 20. The air may then be directed through the connecting tubing to the inlet port 42 of the blanket 40. The air can be directed through the blanket 40 and through the outlet port 44. The air may be exhausted via the outlet port 44 for the on-way system 10 or may be directed through the recirculation tubing 170 for the closed loop system 10. This may provide the heating or cooling effect to the area underlying the patient and the area over the patient in a single airflow path.

The temperature may be slightly different in the different components due to heat lost to the patient in the heating mode or heat gained from the patient in the cooling mode, such that the effect from the blanket 40 (or second component 24) is slightly less than the effect from the surface assembly 14 (or first component 24). An additional heater 160 may be used at the connecting tubing for increasing heat. It is contemplated that the components 24 may be set in sequence in any order (e.g., accessory 40 then surface assembly 14, surface assembly 14 then accessory, multiple accessories 40, multiple temperature regulating/MCM components 24, etc.). Additionally, the temperature effects may be provided concurrently when using multiple pneumatic assembly 26.

Referring to FIG. 13, the heating/MCM system 10 includes the controller 32, which includes a processor 180, a memory 182, and other control circuitry. Instructions or routines 184 are stored in the memory 182 and executable by the processor 180. The memory 182 includes routines 184 that relate to functions of the blower 30, the flow rate valve 36, the heater 160, and the switch valve 38 based on various inputs or protocols initiated by the caregiver. The controller 32 may be a designated controller 32 for the heating/MCM system 10, such as a controller 32 housed in the pneumatic assembly 26, or may be the controller 32 of the patient support apparatus 12. The designated controller 32 may be advantageous for more portable systems 10. The controller 32 may also include communication circuitry 186 for bidirectional wired or wireless communication.

The heating/MCM system 10 includes the sensor assembly 162 communicatively coupled with the controller 32. The sensor assembly 162 may include one or more of a pressure sensor 190, an airflow sensor 192, and a thermal sensor 194. The pressure sensor 190 may sense pressure proximate to the blower 30, such as a discharge pressure at the discharge outlet 136, which is affected by the restriction caused by flow rate valve 36. The airflow sensor 192 may be configured to sense airflow at or downstream of the discharge outlet 136, which can also indicate the restriction setting of the flow rate valve 36. The thermal sensor 194 may sense the temperature of the discharge air at the discharge outlet 136.

The sensor assembly 162 communicates sensed information to the controller 32. The controller 32 may utilize the sensed information to monitor the function of the heating/MCM system 10. The controller 32 may also utilize the sensed information as feedback for controlling the heating/MCM system 10. For example, the thermal sensor 194 can be utilized to monitor the temperature of the discharge air so that the speed of the blower 30 and/or the setting of the flow rate valve 36 can be regulated to increase or decrease the temperature. In such examples, if the thermal sensor 194 is sensing the air is below a predefined temperature or outside a predefined range for heating the patient, the controller 32 may increase the speed or duty cycle of the blower 30 and/or restrict the airflow more with the flow rate valve 36 to generate additional heat and/or activate the heater 160. If the thermal sensor 194 is sensing the air is above a predefined temperature or outside a predefined range, the controller 32 may lower the speed or duty cycle of the blower 30, open the airflow path more by reducing restriction with the flow rate valve 36, and/or deactivate the heater 160.

The blower 30 included in the enclosure 70 with the thermal sensor 194 allows the thermal sensor 194 to be used to monitor the heating/MCM system 10 in real-time. The sensed information from the thermal sensor 194 (i.e., sensed temperature), or the sensor assembly 162, can be used as control feedback, where the PWM of the blower 30 and/or the setting of the flow rate valve 36 can be actively or dynamically adjusted to satisfy the selected or desired output temperature for the patient. Additional temperature or thermal sensors 194 may be disposed in the housing 28 to monitor the temperature of the air drawn into the blower 30 and/or within the treating components of the system 10 (e.g., the surface assembly 14, the blanket 40, other accessories 40 or components 24, etc.) to monitor the temperature of the air flowing through the components proximate to the patient.

The controller 32 may be in communication with a user interface 200, such as a graphical user interface (GUI) on the support apparatus 12 or a remote device (e.g., a phone, a tablet, a wearable device, etc.). The caregiver may input a command into the user interface 200 for controlling the activation, deactivation, or function of the heating/MCM system 10. The input may also relate to a therapy protocol, which can cause adjustment or control of the heating/MCM system 10. For example, the therapy protocol may relate to pressure ulcers, which would cause activation of the MCM cooling function. The therapy protocol may relate to hypothermia, which would cause activation of the treatment-based heating function. In additional examples, the therapy protocol may be complex or multi-faceted, such as for treating pressure ulcers and hypothermia. The controller 32 can execute the routines 184 related to the input protocol or protocols and adjust the heating/MCM system 10 between providing the MCM cooling and moisture wicking effect and the heating effect based on information received from the caregiver input.

Additionally or alternatively, the user interface 200 may be utilized by the caregiver to view information regarding the heating/MCM system 10. Such information may include the temperature of the discharge air, the status or state of the switch valve 38, the restriction setting of the flow rate valve 36, activation status of the heater 160 where included, etc. The caregiver may monitor, adjust, and control the heating/MCM system 10 via the user interface 200.

With reference still to FIG. 13, the controller 32 may be configured to communicate via wired and wireless communication protocols. The controller 32 may be configured to communicate with the user interface 200, a remote server, a local server, etc. Communication with the server or servers may allow for storage of patient information from the controller 32 in an electronic medical record (EMR). In such examples, the information from the heating/MCM system 10, such as time of heating or cooling, temperature of discharge air, etc., may be stored in the EMR of the patient automatically. Further, the controller 32 may retrieve or receive information from various facility systems or from the EMR to adjust the heating/MCM system 10, such as activating or adjusting the blower 30, the flow rate valve 36, the heater 160, and/or the switch valve 38.

In various examples, coupling the heating/MCM system 10 to the support apparatus 12 may provide for wired communication between the heating/MCM system 10 and the support apparatus 12. In such examples, the heating/MCM system 10 and the support apparatus 12 may have mating communication connectors that engage one another to provide wired communication therebetween. Further, as described herein. The heating/MCM system 10, or a portion of the heating/MCM system 10 (e.g., such as the pneumatic assembly 26) may be integrated into the support apparatus 12 and be in wired communication with various components of the support apparatus 12. The wired communication may be via the electrical connectors 62 (FIG. 3).

In additional or alternative configurations, the controller 32 is configured to communicate with remote or external components via a wireless communication network. The communication network may be part of a network of the medical facility, which may include a combination of wired connections (e.g., Ethernet), as well as wireless connections, which may include the wireless communication network. The communication network may include a variety of electronic devices, which may be configured to communicate over various wired or wireless communication protocols. The communication network may include a wireless router through which the remotely accessed devices may be in communication with one another as well as a local server.

The communication network may be implemented via one or more direct or indirect nonhierarchical communication protocols, including but not limited to, Bluetooth®, Bluetooth® low energy (BLE), Thread, Ultra-Wideband, Z-wave, ZigBee, etc. Additionally, the communication network may correspond to a centralized or hierarchal communication network where one or more of the devices communicate via the wireless router (e.g., a communication routing controller). Accordingly, the communication network may be implemented by a variety of communication protocols, including, but not limited to, global system for mobile communication (GSM), general packet radio services, code division multiple access, enhanced data GSM environment, fourth generation (4G) wireless, fifth generation (5G) wireless, Wi-Fi, world interoperability for wired microwave access (WiMAX), local area network, Ethernet, etc. By flexibly implementing the communication network, the various devices and servers may be in communication with one another directly via the wireless communication network, or cellular data connection.

In certain aspects, the controller 32 may be capable of communicating wirelessly via a wireless communication module. The wireless communication module generally communicates via an SPI link with circuitry associated with the heating/MCM system 10 and/or the support apparatus 12 (e.g., the communication circuitry 186) and the wireless 802.11 link with wireless access points. The wireless access points are generally coupled to Ethernet switches via 802.3 links. It is contemplated that the wireless communication modules may communicate with the wireless access points via any of the wireless protocols disclosed herein. Additionally or alternatively, the Ethernet switches may generally communicate with Ethernet via an 802.3 link. Ethernet is also in communication with the local server, allowing information and data to be communicated to and from the controller 32.

With reference to FIG. 14, the heating/MCM system 10 is included in or used in conjunction with the patient support apparatus 12. In various aspects, the heating/MCM system 10 may be a separate assembly including the surface assembly 14 and/or another temperature regulating/MCM components 24 for supporting the patient and the pneumatic assembly 26 for generating the airflow through the surface assembly 14 or another temperature regulating/MCM components 24, respectively. In such examples, the heating/MCM system 10 may be selectively positioned on and removed from various support apparatuses 12. Additionally, the blanket 40, or other accessories 40, can be positioned over the patient supported on the surface assembly 14 or the support apparatus 12 and can be moved with the patient between different support apparatuses 12. In additional or alternative examples, the heating/MCM system 10 may be at least partially integrated into the support apparatus 12. In such examples, the surface assembly 14 may be positioned on the support apparatus 12 and coupled to the pneumatic assembly 26 or the blanket 40 positioned over the patient and coupled to the pneumatic assembly 26, where the pneumatic assembly 26 is (or multiple assemblies 26 are) installed or integrated into the support apparatus 12.

In the example illustrated in FIG. 14, the surface assembly 14 is illustrated with the support apparatus 12 configured as a medical bed and the blanket 40 is positioned over the surface assembly 14. In the example with the medical bed, the bed includes a frame assembly 206 with a base frame 208 coupled to an upper frame 210. The upper frame 210 is adjustable relative to the base frame 208 (e.g., raise, lower, tilt, etc.). Additionally, the upper frame 210 generally includes multiple segments that are independently adjustable relative to one another, allowing the upper frame 210 to articulate between various positions (e.g., an elevated head region, elevated foot region, etc.). The medical bed includes actuation assemblies for adjusting the upper frame 210 and the segments. The base frame 208 includes wheels 212 for moving the medical bed relative to an underlying floor surface. The medical bed also includes siderails 214, which are generally adjustable between a raised position in a lowered position. The GUI (e.g., the user interface 200) is often included on one of the siderails 214.

In such examples, the pneumatic assembly 26 may be coupled to or integrated with the frame assembly 206 of the medical bed. The frame assembly 206 may include a receiving space for receiving and holding the enclosure 70. The tubing 50, 170 and electrical connectors 62 may extend from the enclosure 70 to engage the surface assembly 14, blanket 40, other temperature regulating/MCM components 24, and/or other components of the medical bed.

While illustrated as the medical bed, it is also contemplated that the heating/MCM system 10 may be used with or integrated into various support apparatuses 12 including stretchers, surgical tables, med-surg beds, high-acuity surfaces, etc. This allows the heating/MCM system 10 to be used in multiple care settings, such as the emergency department (ED), during transport, etc. For example, the heating/MCM system 10 can be used in the ED for treating hypothermia, used during a procedure performed on a stretcher, and for regulating body temperature during post-operative recovery. This provides increased care and flexibility in care for the patient. Moreover, the components 24 included in the system 10 may differ based on the type of support apparatus 12 used or the unit in which the patient is being treated.

Referring again to FIGS. 1-14, with each support apparatus 12, the heating/MCM system 10 may have multiple configurations. The heating/MCM system 10 may include one or more of the surface assembly 14, the blanket 40, and/or other temperature regulating/MCM components 24 positioned on the support apparatus 12 or the patient, as well as the pneumatic assembly 26 removable from the support apparatus 12. The heating/MCM system 10 may include the temperature regulating/MCM components 24 (e.g., the surface assembly 14, the blanket 40, or other accessories 40) positioned on the support apparatus 12 and the pneumatic assembly 26 integrated into the support apparatus 12. The heating/MCM system 10 may also include both the temperature regulating/MCM components 24 of the heating/MCM system 10 and the pneumatic assembly 26 integrated into the support apparatus 12. For example, the upper frame 210 or upper deck of the support apparatus 12 may include an integrated surface assembly 14 or selectively receive the surface assembly 14. For each configuration of the surface assembly 14, the surface assembly 14 may include the coverlet 52, the mattress 54, the mattress 54 with the MCM layer, or the MCM-based mattress 54 as described herein.

The heating/MCM system 10 can support the patient or extend over the patient while providing additional comfort and therapies with the temperature effect of the air being directed through the surface assembly 14 or the other component 24 by the pneumatic assembly 26. The heating/MCM system 10 can switch between heating and cooling functions to provide increased and/or customized care for the patient. In this way, the surface assembly 14 and other temperature regulating/MCM components 24 can be used for both microclimate management and heating. For example, the heating/MCM system 10 can provide a warmer surface for hypothermia patients by directing warmed or heated air through the temperature regulating/MCM components 24 (such as the surface assembly 14 or blanket 40) and also provide a cooler surface for patients with a longer stay by directing cool or ambient air through the temperature regulating/MCM components 24 (e.g., surface assembly 14 or blanket 40) to keep the skin of the patient cool and dry. In this way, the heating/MCM system 10 can warm or cool the skin of the patient in contact with the temperature regulating/MCM components 24 (e.g., surface assembly 14 or accessory/blanket 40), which can be accomplished by adjusting the speed of the blower 30 with the electrical motor 150, adjusting the duty cycle of the blower 30, adjusting the restriction at the intake 34 of the blower 30, adjusting the restriction at the discharge outlet 136, and/or adjusting the heater 160.

The pneumatic assembly 26 includes the blower 30 or both the blower 30 and the flow rate valve 36, which are controlled to generate heat to warm the discharge air. The discharge air may not be warmed significantly to direct generally ambient air through the temperature regulating/MCM components 24 to cool the skin of the patient and whisk moisture. The discharge air can be warmed slightly (less than about 10° C.) relative to ambient to provide additional comfort to the patient. The discharge air can also be warmed a more significant amount or degree (between about 10° C. and about 20° C.) to provide heat-based therapy to the patient.

The adjustments to the blower 30 and the flow rate valve 36 may independently generate similar amounts of heat based on a level of adjustment. The combination of the blower 30 and the flow rate valve 36 may generate more heat than the blower 30 and the flow rate valve 36 independently. Further, the heater 160 may be utilized for the heating or to contribute to the heating of the discharge air.

Moreover, the recirculation of the air may assist with heating the air by drawing pre-warmed air into the pneumatic assembly 26. Different amounts of heat can be generated by adjusting the operating speed of the blower 30, the duty cycle of the blower 30, the restriction level of the flow rate valve 36, the activation of the blower 30, and/or the position or state of the switch valve 38. Accordingly, one or more components or features of the heating/MCM system 10 may contribute to the temperature effect, which can provide more customizable treatment for the patient.

The heating/MCM system 10 purposefully generates additional heat to provide different functions in the heating/MCM system 10. This allows the same heating/MCM system 10 to provide both cooling and heating for the patient. In conventional approaches, strategies for patient warming in the perioperative space are often additional, separate components or equipment. For example, conventional approaches include blowing warm air across the patient, which can increase the risk of infection, using separate heated blankets, which can be difficult to repurpose or reuse, using regular blankets, which can be difficult to regulate in the operating room and under anesthesia, and circulating warm water through a pad, which utilizes a specific accessory. The heating/MCM system 10 disclosed herein can be integrated in or with the support apparatus 12 as opposed to being additional accessories or may operate in conjunction with the support apparatus 12. The disclosed heating/MCM system 10 can also provide both cooling and heating with the same components and can selectively switch between heating and cooling based on the operation of components included in the heating/MCM system 10.

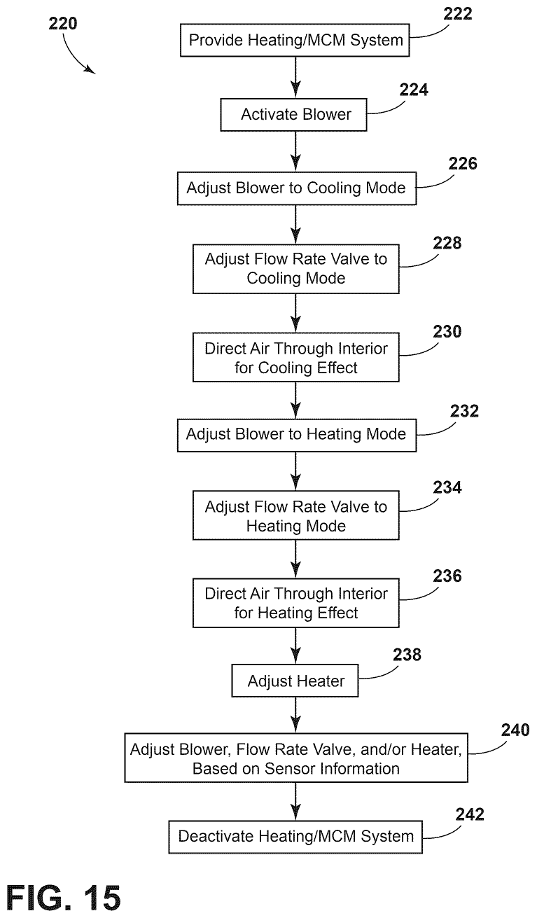

With reference to FIG. 15, as well as FIGS. 1-14, a method 220 of controlling the heating/MCM system 10 includes step 222 of providing the heating/MCM system 10. The heating/MCM system 10 may be coupled to the frame assembly 206, and the temperature regulating/MCM component 24, such as the surface assembly 14 or the blanket 40, is positioned on the support apparatus 12. The pneumatic assembly 26 and the component 24 are fluidly coupled via at least the connecting inlet tubing 50.

In step 224, the blower 30 may be activated to generate the airflow through component 24, which may be the surface assembly 14, the blanket 40, etc. The activation may be in response to an input communicated by the caregiver via the user interface 200 or retrieved from the server or an electronic medical record (EMR) for the patient by the controller 32. The input may relate to a therapy or treatment protocol. In step 226, the blower 30 is adjusted for the cooling mode. In the cooling mode, the blower 30 is adjusted to the first operating speed, which is generally a lower operating speed to produce the airflow without generating a significant amount of heat. In step 228, in examples with the flow rate valve 36, the flow rate valve 36 may be adjusted to the cooling mode. In the cooling mode, the flow rate valve 36 is adjusted to a lower restrictive setting, allowing a higher flow rate for the discharge air. The lower restrictive setting results in the blower 30 generating minimal or no additional heat.

In step 230, the airflow is directed through component 24. For example, the airflow may be directed through the surface assembly 14 from the inlet port 18, through the interior, and exits the surface assembly 14 through the outlet port 20. In certain aspects, when the air is being directed through the interior, the air is being directed through the spacer material 22. Generally, in step 230, the air exits the surface assembly 14 and is exhausted to the external atmosphere. For use with the blanket 40, the airflow can be directed through the blanket 40 from the inlet port 42, through the interior or spacer material 48, and exits through the outlet port 44. The airflow generally performs the MCM cooling function based on the lower speed of the blower 30 and/or the lower restrictive setting of the flow rate valve 36 to generate the cooling effect with the airflow. The cooling effect is used to cool the skin of the patient and wick away moisture for microclimate management on patients in longer stays, that are susceptible to pressure ulcers, etc.

In step 232, the blower 30 is adjusted to the second operating speed that is higher than the first operating speed. In this way, the PWM for the blower 30 is greater from a greater amount of electrical energy being used. The greater amount of electrical energy results in a greater amount of heat caused by the blower 30 within the housing 28. The configuration of the housing 28 and the enclosure 70 assist with retaining the heat proximate to the blower 30 to be captured by the blower 30.

In step 234, the flow rate valve 36 can be adjusted to the higher restrictive setting, which reduces the flow rate of the discharge air and causes an increase in pressure and an increase in heat generation. It is contemplated that the flow rate valve 36 may be adjusted after the blower 30 in step 232 or that step 232 may be omitted. When step 232 is omitted, the blower 30 may operate at the same speed as when operating in the cooling mode with the heat generation being caused by the flow rate valve 36 restriction.

In step 236, the heat is captured by the blower 30 and directed as the heated airflow through component 24, such as the surface assembly 14, blanket 40, etc. The air drawn into the enclosure 70 is drawn through the interior inlet tubing 130 and into the housing 28 proximate to the base of the blower 30. The air is then drawn along the height of the blower 30, capturing heat or warming to be directed by the blower 30. The warmed or heated air is directed through the spacer material 22, 48, warming the skin of the patient, and then exhausted through the outlet port 20, 44. Additionally, in examples with the heater 160, the controller 32 may selectively activate and deactivate the heater 160 in step 238 to generate the warming effect for the patient.

In step 240, the controller 32 receives sensed information from the sensor assembly 162 and adjusts the pneumatic assembly 26 based on the sensed information. The sensed information may include one or more of pressure information, temperature information, and flow rate information. The controller 32 may actively, dynamically, and in real-time adjust the pneumatic assembly 26 to generate the discharge air with the flow rate and the temperature for the selected operating mode (e.g., cooling mode or heating mode). In step 242, the heating/MCM system 10 may be deactivated.

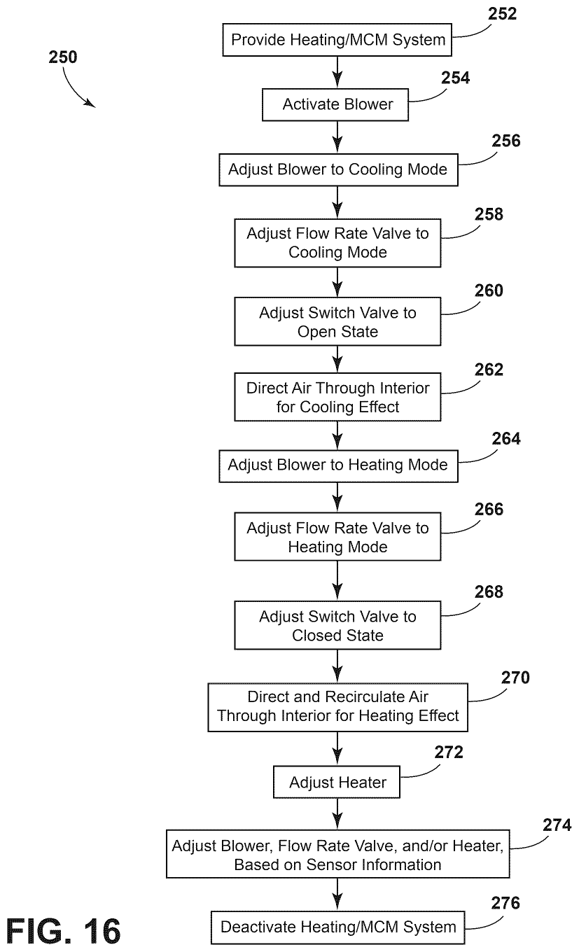

With reference to FIG. 16, as well as FIGS. 1-15, a method 250 of controlling the heating/MCM system 10 that can recirculate the air includes step 252 of providing the heating/MCM system 10. The heating/MCM system 10 may be coupled to the frame assembly 206, and the component 24, such as the surface assembly 14, blanket 40, or other accessory 40, is positioned on the support apparatus 12. The pneumatic assembly 26 and temperature regulating/MCM component 24, such as the surface assembly 14 or blanket 40, are fluidly coupled via the connecting inlet tubing 50 and the recirculation tubing 170. Similar to step 224 in method 220, in step 254 in method 250, the blower 30 may be activated to generate the airflow through the surface assembly 14, the blanket 40, or other system component 24. The activation may be in response to an input communicated by the caregiver via the user interface 200 or retrieved from the server or the EMR for the patient by the controller 32. The input may relate to a therapy or treatment protocol.

In step 256, the blower 30 is adjusted for the cooling mode. In the cooling mode, the blower 30 is adjusted to the first, lower operating speed to produce the airflow without generating a significant amount of heat. Generally, the air is drawn in through the switch valve 38 and is ambient air. In step 258, in examples with the flow rate valve 36, the flow rate valve 36 may be adjusted to the cooling mode. In the cooling mode, the flow rate valve 36 is adjusted to a lower restrictive setting, allowing a higher flow rate for the discharge air and resulting in the blower 30 generating minimal or no additional heat.

In step 260, the switch valve 38 is adjusted to the opened or exhaust position. In step 262, when using the surface assembly 14, the airflow is directed through the surface assembly 14 from the inlet port 18, through spacer material 22, and exits the surface assembly 14 through the outlet port 20. In step 262, the air exits the surface assembly 14, and flows through the recirculation tubing 170, to be exhausted to the external area via the switch valve 38. When using the blanket 40, airflow is directed through the blanket 40 from the inlet port 42, through the spacer material 48, and exits the blanket 40 through the outlet port 44 to the recirculation tubing 170. The airflow generally performs the MCM cooling function based on the lower speed of the blower 30 and/or the lower restrictive setting of the flow rate valve 36 to generate the cooling effect with the airflow. The cooling effect is used to cool the skin of the patient and wick away moisture for microclimate management on patients in longer stays, that are susceptible to pressure ulcers, etc.

In step 264, the blower 30 is adjusted to the second, higher operating speed for the heating mode. In this way, the PWM for the blower 30 is greater, which results in a greater amount of heat caused by the blower 30 within the housing 28. The configuration of the housing 28 and the enclosure 70 assist with retaining the heat proximate to the blower 30 to be captured by the blower 30.

In step 266, the flow rate valve 36 can be adjusted to the higher restrictive setting, to cause an increase in pressure and an increase in heat generation. It is contemplated that the flow rate valve 36 may be adjusted after the blower 30 in step 264 or that step 264 may be omitted. When step 264 is omitted, the blower 30 may operate at the same speed as when operating in the cooling mode with the heat generation being caused by the flow rate valve 36 restriction.

In step 268, the switch valve 38 is adjusted to the closed or recirculation position. In step 270, the heat is captured by the blower 30 and directed as the heated airflow through the surface assembly 14, blanket 40, or other system component 24. The air drawn into the enclosure 70 is drawn through the interior inlet tubing 130 and into the housing 28 proximate to the base or base portion of the blower 30. Initially, this air may be ambient air. After the switch valve 38 adjusts to the recirculation position, the air drawn through the interior inlet tubing 130 may be recirculated air from the system component 24, such as the surface assembly 14 or blanket 40. The air is drawn along the height of the blower 30, capturing heat or warming to be directed by the blower 30. The warmed or heated air is directed through the spacer material 22, 48, warming the skin of the patient, and then exiting through the outlet port 20, 44.

Additionally, in examples with the heater 160, the controller 32 may selectively activate and deactivate the heater 160 in step 272 to generate the warming effect for the patient. The air exits the surface assembly 14 and is redirected to the pneumatic enclosure 70 via the recirculation tubing 170 and the switch valve 38 to again be directed through the surface assembly 14 or other system component 24 for the heating mode.

In step 274, the controller 32 receives sensed information from the sensor assembly 162 and adjusts the pneumatic assembly 26 based on the sensed information. The sensed information may include one or more of pressure information, temperature information, and flow rate information. The controller 32 may actively, dynamically, and in real-time adjust the pneumatic assembly 26 to generate the discharge air with the flow rate and the temperature for the selected operating mode (e.g., cooling mode or heating mode). In step 276, the heating/MCM system 10 may be deactivated. The steps of the methods may be performed in any order, concurrently, omitted, repeated, etc. without departing from the teachings herein.

Use of the present device and system may provide for a variety of advantages. For example, the temperature regulating/MCM component 24, such as the MCM layer or MCM coverlet 52, blanket 40, or other accessory 40, that provides cooling and microclimate management can be turned into a heater. Additionally, the amount of heat generated can be controlled by changing the speed (in rotations per minute or RPM) of the blower 30 and/or the level of restriction caused in the airflow by the flow rate valve 36. Moreover, the heating/MCM system 10 can also include the resistive heater 160 to further control the air temperature, as well as provide an initial “jump start” for heating the heating/MCM system 10. Also, the heating/MCM system 10 may include the sensor assembly 162 that can provide real-time information and, consequently, result in real-time control of the heating/MCM system 10 to provide the selected temperature effect. Further, the sensor assembly 162 can provide active monitoring of the heating/MCM system 10, resulting in active and dynamic adjustment of the heating/MCM system 10. Moreover, the heating/MCM system 10 can be set to a predefined temperature or switch between predefined temperatures. Additional benefits or advantages may be realized and/or achieved.

The system disclosed herein is further summarized in the following paragraphs and is further characterized by combinations of any and all of the various aspects described therein.

According to one aspect of the present disclosure, a temperature regulating and microclimate management surface system for a patient support apparatus includes a surface assembly including an outer covering having an inlet port and an outlet port and a spacer material disposed within an interior of the outer covering. A pneumatic assembly is in fluid communication with the inlet port. The pneumatic assembly includes a housing and a blower disposed within the housing. The blower is configured to direct air through the spacer material via the inlet port. A controller is communicatively coupled to the pneumatic assembly. The controller is configured to activate the blower at a first operating speed to direct the air through the surface assembly and generate a cooling effect. The controller is also configured to generate a heating effect through the surface assembly by at least one of: adjusting the blower to a higher second operating speed to generate heat in the housing and warm the air, where the blower is configured to intake heated air and direct the heated air through the surface assembly; restricting a discharge flow rate of the blower via a flow rate valve to generate the heat in the housing and direct the heated air through the surface assembly; and recirculating exiting heated air from the outlet port to the pneumatic assembly to be redirected through the surface assembly.

According to another aspect of the present disclosure, a controller is configured to generate a heating effect by both adjusting a blower to a second higher operating speed and restricting a discharge flow rate of the blower.

According to another aspect of the present disclosure, a blower is configured to draw air along a height of the blower to warm the air with the heat to produce heated air.

According to another aspect of the present disclosure, a switch valve is in fluid communication with a pneumatic assembly and a surface assembly. The switch valve is configured to exhaust exiting heated air from the surface assembly when in an opened state in a cooling mode and recirculate the exiting heated air when in a closed state in a heating mode.

According to another aspect of the present disclosure, an outer covering and a spacer material form a coverlet. A surface assembly includes the coverlet coupled to a mattress.

According to another aspect of the present disclosure, a surface assembly includes a mattress with a spacer material forming a microclimate management layer within the mattress.

According to another aspect of the present disclosure, air flowing through a surface assembly and generating a cooling effect has a temperature of 5° C. or less above ambient temperature.

According to another aspect of the present disclosure, heated air flowing through a surface assembly and generating a heating effect has a temperature of between 10° C. and 20° C. above ambient temperature.

According to another aspect of the present disclosure, exiting heated air recirculated to a pneumatic assembly has a temperature above ambient temperature.

According to another aspect of the present disclosure, a sensor assembly is communicatively coupled to a controller. The controller is configured to adjust at least one of a blower and a flow rate valve in response to sensed information from the sensor assembly.

According to another aspect of the present disclosure, a sensor assembly includes a thermal sensor, and sensed information includes a temperature of discharge air from a blower.

According to another aspect of the present disclosure, a resistive heater is communicatively coupled to a controller. The resistive heater is configured to generate heat to warm discharge air from the blower.

According to another aspect of the present disclosure, a pneumatic assembly is coupled to a frame assembly, and a surface assembly is positioned on the frame assembly.

According to another aspect of the present disclosure, a pneumatic assembly is integrated into a frame assembly, and a surface assembly is positioned on the frame assembly.

According to another aspect of the present disclosure, a blanket fluidly coupled with the pneumatic assembly, wherein the controller is configured to direct the air through the blanket and generate the cooling effect and generate the heating effect through the blanket.

According to another aspect of the present disclosure, a heating and cooling surface system includes a surface assembly including an outer covering and a spacer material disposed within an interior of the outer covering. A pneumatic assembly is in fluid communication with the interior. The pneumatic assembly includes a housing. A blower is disposed within the housing, and the blower has an intake and a discharge outlet. A flow rate valve is operably coupled to the discharge outlet of the blower. The flow rate valve is configured to adjust a discharge flow rate of the blower. A controller is communicatively coupled to the blower and the flow rate valve. The controller is configured to adjust the flow rate valve to a first restriction setting for a cooling effect with airflow directed through the surface assembly by the blower and adjust the flow rate valve to a second restriction setting for a heated airflow to be directed through the surface assembly by the blower for a heating effect. The second restriction setting is higher than the first restriction setting to cause a greater restriction on the discharge flow rate from the blower to generate heat that is captured by the blower and produce the heating effect with the heated airflow.

According to another aspect of the present disclosure, a controller is configured to operate the blower at a single operating speed when the flow rate valve is at the first restriction setting and the second restriction setting.

According to another aspect of the present disclosure, a controller is configured to adjust a blower to a first operating speed when a flow rate valve is at a first restriction setting and to a second higher operating speed when the flow rate valve is at a second restriction setting.

According to another aspect of the present disclosure, an inlet opening of a housing is defined proximate to a base of a blower. An intake of the blower is defined at a top of the blower.

According to another aspect of the present disclosure, a blower is configured to draw air from an inlet opening and along a height of the blower to capture heat generated by the blower and warm the air to be directed through a surface assembly as a heated airflow for a heating effect.

According to another aspect of the present disclosure, a controller is configured to actively adjust a flow rate valve between a first restriction setting and a second restriction setting to decrease and increase heat in discharge air from a blower for a cooling effect and a heating effect, respectively.

According to another aspect of the present disclosure, a thermal sensor is communicatively coupled to a controller. The controller is configured to adjust a flow rate valve in response to sensed temperature of discharge air of a blower received from the thermal sensor.

According to another aspect of the present disclosure, a heater is communicatively coupled to a controller. The controller is configured to selectively activate the heater to increase a temperature of discharge air from a blower in response to sensed temperature from a thermal sensor being below a predefined temperature. The controller is configured to selectively deactivate the heater to decrease the temperature of the discharge air in response to the sensed temperature from the thermal sensor being above a predefined temperature.

According to another aspect of the present disclosure, a heating and cooling surface system includes a surface assembly including an outer covering and a spacer material disposed within an interior of the outer covering. A pneumatic assembly is in fluid communication with the interior. The pneumatic assembly includes an enclosure operably coupled to the interior of the outer covering via inlet tubing and recirculation tubing. A blower is disposed within the enclosure, and the blower is configured to direct an airflow through the spacer material. A switch valve is operably coupled to the recirculation tubing and the interior of the enclosure. A controller is communicatively coupled to the blower and the switch valve. The controller is configured to activate the blower at a first operating speed to generate a cooling effect with the airflow through the spacer material for a cooling mode, activate the blower at a second operating speed to generate a heating effect with the airflow through the spacer material for a heating mode, the second operating speed being greater than the first operating speed to generate heat captured within the airflow, and adjust the switch valve to a closed state in the heating mode to redirect the airflow from the surface assembly to the blower.

According to another aspect of the present disclosure, a controller is configured to adjust a switch valve to an opened state to exhaust air from a surface assembly when in a cooling mode.

According to another aspect of the present disclosure, a housing is within an enclosure, and a blower is disposed within the housing. An inlet opening of the housing is defined proximate to a base of a blower. The intake of the blower is defined at a top of the blower.

According to another aspect of the present disclosure, a blower is configured to draw air from an inlet opening and along a height of the blower to capture heat generated by the blower and warm the air to be directed through the surface assembly for the heating effect.

According to another aspect of the present disclosure, a pneumatic enclosure includes a flow rate valve operably coupled with a discharge outlet of the blower.

According to another aspect of the present disclosure, a controller is configured to adjust a flow rate valve to a first restriction setting in a cooling mode and adjust the flow rate valve to a second restriction setting in a heating mode. The second restriction setting is greater than the first restriction setting to reduce a discharge flow rate of discharge air from the blower and, consequently, cause the heat to be generated for the heating effect.

According to another aspect of the present disclosure, a blower includes an electrical motor. More electrical energy is provided to the electrical motor in a heating mode to generate heat compared to the electrical energy provided to the electrical motor in a cooling mode.

According to another aspect of the present disclosure, a thermal sensor is communicatively coupled to a controller. A controller is configured to adjust a flow rate valve in response to sensed temperature of discharge air from a blower received from the thermal sensor.

According to another aspect of the present disclosure, a heater is communicatively coupled to a controller. The controller is configured to selectively activate the heater to increase a temperature of discharge air from the blower in response to sensed temperature from a thermal sensor being below a predefined temperature. The controller is configured to selectively deactivate the heater to decrease the temperature of the discharge air in response to the sensed temperature from the thermal sensor being above a predefined temperature.

According to another aspect of the present disclosure, a heating and cooling surface system includes an outer covering. A spacer material is disposed within an interior of the outer covering. A pneumatic assembly is in fluid communication with the interior. The pneumatic assembly includes a housing operably coupled to the interior of the outer covering via inlet tubing. The housing defines an inlet opening. A blower is disposed within the housing. The blower has an intake at an upper portion and a discharge outlet. The inlet opening is defined proximate to a base portion of the blower. A thermal sensor is configured to sense a temperature of air discharged from the blower. A controller is communicatively coupled to the blower. The controller is configured to activate the blower at a first operating speed to generate a cooling effect with an airflow directed through the spacer material by the blower, activate the blower at a higher second operating speed to generate heat in the housing, where the blower is configured to draw the air from the inlet opening and along a height of the blower to warm the air with the heat in the housing and produce heated air. The blower is configured to direct the heated air through the spacer material to, consequently, generate a heating effect with the airflow through the spacer material. The controller is configured to actively adjust an operating speed of the blower in response to a sensed temperature received from the thermal sensor.

According to another aspect of the present disclosure, a controller is configured to lower an operating speed of a blower when a sensed temperature is above a predefined temperature. A controller is configured to increase the operating speed of the blower when the sensed temperature is below a predefined temperature.

According to another aspect of the present disclosure, a pneumatic assembly includes a flow rate valve operably coupled with a discharge outlet of a blower. A controller is configured to adjust the flow rate valve to a first restriction setting for a cooling effect and adjust the flow rate valve to a second restriction setting for a heating effect. A second restriction setting being greater than the first restriction setting to reduce a discharge flow rate from a discharge outlet of the blower and, consequently, cause heat to be generated by the blower for the heating effect.

According to another aspect of the present disclosure, connecting inlet tubing fluidly coupled a pneumatic assembly and an inlet port of an outer covering. Recirculating tubing fluidly couples the pneumatic assembly and an outlet port of the outer covering.

According to another aspect of the present disclosure, a pneumatic assembly includes a switch valve. The switch valve is in an opened state for a cooling effect to exhaust air from recirculation tubing. The switch valve is in a closed state for a heating effect to redirect heated air from the recirculation tubing to the blower.

According to another aspect of the present disclosure, a controller is configured to selectively activate and deactivate a heater in response to a sensed temperature.

According to another aspect of the present disclosure, a surface system for temperature regulation and microclimate management of a patient includes a surface assembly including an outer covering and a spacer material disposed within an interior of the outer covering. A pneumatic assembly is in fluid communication with the interior. The pneumatic assembly includes a housing. A blower is disposed within the housing. The blower has an intake and a discharge outlet. A flow rate valve is operably coupled to the discharge outlet of the blower. The flow rate valve is configured to adjust a discharge flow rate of the blower. A controller is communicatively coupled to the blower and the flow rate valve. The controller is configured to adjust at least one of an operating speed of the blower and a restriction setting of the flow rate valve to selectively generate a cooling effect and a heating effect with airflow directed by the blower through the surface assembly.

According to another aspect of the present disclosure, a controller is configured to maintain an operating speed of a blower and adjust a restriction setting of a flow rate valve to increase restriction to a discharge flow rate of discharge air from the blower in a heating mode to generate heat for a heating effect.

According to another aspect of the present disclosure, a controller is configured to increase an operating speed of a blower and adjust a restriction setting of a flow rate valve to increase restriction to a discharge flow rate of discharge air from the blower in a heating mode to generate heat for a heating effect.

According to another aspect of the present disclosure, a controller is configured to increase the operating speed of the blower to generate heat and maintain the restriction setting of the flow rate valve in a heating mode to generate heat for the heating effect.

According to another aspect of the present disclosure, a controller is configured to decrease an operating speed of a blower and adjust a restriction setting of a flow rate valve to decrease restriction to a discharge flow rate of discharge air from the blower in a cooling mode to generate a cooling effect.

According to another aspect of the present disclosure, a method of controlling a temperature regulating surface system includes: activating a blower in a pneumatic assembly to generate an airflow through a surface assembly; adjusting the blower to a first operating speed in a cooling mode; adjusting a flow rate valve operably coupled to the blower to a first restriction setting for discharge air from the blower in the cooling mode, where the blower at the first operating speed and the flow rate valve at the first restriction setting generate a cooling effect with the airflow through the surface assembly; adjusting the blower to a second operating speed higher than the first operating speed to generate heat in the airflow in a heating mode; and adjusting the flow rate valve to a second restriction setting for the discharge air that is more restrictive than the first restriction setting to generate the heat in the airflow, where adjusting the blower to the second operating speed and the flow rate valve to the second restriction setting, consequently, generates a heating effect with the airflow through the surface assembly.

According to one aspect of the present disclosure, a method of controlling a temperature regulating surface system includes: activating a blower of a pneumatic assembly to generate an airflow through a surface assembly; adjusting the blower to a first operating speed in a cooling mode to generate a cooling effect with the airflow; adjusting a switch valve to an exhaust state when in the cooling mode to direct exiting air from the surface assembly to an external area; adjusting the blower to a second operating speed higher than the first operating speed to generate heat in the airflow and, consequently, generate a heating effect with the airflow in a heating mode; and adjusting the switch valve to a closed state when in the heating mode to redirect the exiting air from the surface assembly to the pneumatic assembly.

According to another aspect, a temperature regulating and microclimate management surface system for a patient support apparatus includes a surface assembly including an outer covering having an inlet port and an outlet port. A pneumatic assembly is in fluid communication with the inlet port. The pneumatic assembly includes a housing and a blower disposed within the housing. The blower is configured to direct air through the surface assembly via the inlet port. A controller is communicatively coupled to the pneumatic assembly. The controller is configured to activate the blower at a first operating speed to direct the air through the surface assembly and generate a cooling effect and configured to generate a heating effect through the surface assembly by at least one of: adjusting the blower to a higher second operating speed to generate heat in the housing and warm the air, wherein the blower is configured to intake heated air and direct the heated air through the surface assembly; restricting a discharge flow rate of the blower via a flow rate valve to generate the heat in the housing and direct the heated air through the surface assembly; and recirculating exiting heated air from the outlet port to the pneumatic assembly to be redirected through the surface assembly.

According to another aspect, a heating and cooling surface system includes a surface assembly defining an interior. A pneumatic assembly is in fluid communication with the interior. The pneumatic assembly includes a housing operably coupled to the interior of the outer covering via inlet tubing. The housing defines an inlet opening. A blower is disposed within the housing. The blower has an intake at an upper portion and a discharge outlet, wherein the inlet opening is defined proximate to a base portion of the blower. A thermal sensor is configured to sense a temperature of air discharged from the blower. A controller is communicatively coupled to the blower. The controller is configured to activate the blower at a first operating speed to generate a cooling effect with an airflow directed through the interior by the blower. Activate the blower at a higher second operating speed to generate heat in the housing. The blower is configured to draw the air from the inlet opening and along a height of the blower to warm the air with the heat in the housing and produce heated air. The blower is configured to direct the heated air through the interior to, consequently, generate a heating effect with the airflow of the heated air through the interior and actively adjust an operating speed of the blower in response to a sensed temperature received from the thermal sensor.

According to one aspect of the present disclosure, a temperature regulating and microclimate management system for a patient support apparatus includes a blanket including an outer covering having an inlet port and an outlet port and a spacer material disposed within an interior of the outer covering. A pneumatic assembly is in fluid communication with the inlet port. The pneumatic assembly includes a housing and a blower disposed within the housing. The blower is configured to direct air through the spacer material via the inlet port. A controller is communicatively coupled to the pneumatic assembly. The controller is configured to activate the blower at a first operating speed to direct the air through the blanket and generate a cooling effect. The controller is also configured to generate a heating effect through the blanket by at least one of: adjusting the blower to a higher second operating speed to generate heat in the housing and warm the air, where the blower is configured to intake heated air and direct the heated air through the blanket; restricting a discharge flow rate of the blower via a flow rate valve to generate the heat in the housing and direct the heated air through the blanket; and recirculating exiting heated air from the outlet port to the pneumatic assembly to be redirected through the blanket.

According to another aspect of the present disclosure, a controller is configured to generate a heating effect by both adjusting a blower to a second higher operating speed and restricting a discharge flow rate of the blower.

According to another aspect of the present disclosure, a blower is configured to draw air along a height of the blower to warm the air with the heat to produce heated air.

According to another aspect of the present disclosure, a switch valve is in fluid communication with a pneumatic assembly and a blanket. The switch valve is configured to exhaust exiting heated air from the blanket when in an opened state in a cooling mode and recirculate the exiting heated air when in a closed state in a heating mode.

According to another aspect of the present disclosure, an outer covering and a spacer material form a coverlet. A blanket includes the coverlet coupled to a mattress.

According to another aspect of the present disclosure, a blanket includes a mattress with a spacer material forming a microclimate management layer within the mattress.

According to another aspect of the present disclosure, air flowing through a blanket and generating a cooling effect has a temperature of 5° C. or less above ambient temperature.

According to another aspect of the present disclosure, heated air flowing through a blanket and generating a heating effect has a temperature of between 10° C. and 20° C. above ambient temperature.

According to another aspect of the present disclosure, exiting heated air recirculated to a pneumatic assembly has a temperature above ambient temperature.

According to another aspect of the present disclosure, a sensor assembly is communicatively coupled to a controller. The controller is configured to adjust at least one of a blower and a flow rate valve in response to sensed information from the sensor assembly.

According to another aspect of the present disclosure, a sensor assembly includes a thermal sensor, and sensed information includes a temperature of discharge air from a blower.

According to another aspect of the present disclosure, a resistive heater is communicatively coupled to a controller. The resistive heater is configured to generate heat to warm discharge air from the blower.

According to another aspect of the present disclosure, a pneumatic assembly is coupled to a frame assembly, and a blanket is positioned on the frame assembly.

According to another aspect of the present disclosure, a pneumatic assembly is integrated into a frame assembly, and a blanket is positioned on the frame assembly.

According to one aspect of the present disclosure, a heating and microclimate management system for a patient support apparatus includes a temperature regulating component including an outer covering having an inlet port and an outlet port and a spacer material disposed within an interior of the outer covering. A pneumatic assembly is in fluid communication with the inlet port. The pneumatic assembly includes a housing and a blower disposed within the housing. The blower is configured to direct air through the spacer material via the inlet port. A controller is communicatively coupled to the pneumatic assembly. The controller is configured to activate the blower at a first operating speed to direct the air through the temperature regulating component and generate a cooling effect. The controller is also configured to generate a heating effect through the temperature regulating component by at least one of: adjusting the blower to a higher second operating speed to generate heat in the housing and warm the air, where the blower is configured to intake heated air and direct the heated air through the temperature regulating component; restricting a discharge flow rate of the blower via a flow rate valve to generate the heat in the housing and direct the heated air through the temperature regulating component; and recirculating exiting heated air from the outlet port to the pneumatic assembly to be redirected through the temperature regulating component.

According to another aspect of the present disclosure, a temperature regulating component includes at least one of a surface assembly and a blanket. A means for regulating temperature includes a support means with a covering means having a means for receiving air and a means for expelling air. An airflow spacer means is disposed within an interior of the covering means. A means for directing airflow is in fluid communication with the means for receiving air. The means for directing airflow includes a housing means and a means for generating air, which is configured to generate and direct the air through the airflow spacer means via the means for receiving air. A control means is communicatively coupled to the means for directing airflow. The control means to activate the means for generating air at a first operating speed to direct the air through the support means and generate a cooling effect and configured to generate a heating effect through the support means by at least one of adjusting the means for generating air to a higher second operating speed to generate heat in the housing means and warm the air, restricting a discharge flow rate of the means for generating air via a means for restricting flow rate to generate the heat in the housing means, and recirculating exiting heated air from the means for expelling air to the means for directing air to be redirected through the support means.

It will be understood by one having ordinary skill in the art that construction of the described disclosure and other components is not limited to any specific material. Other exemplary embodiments of the disclosure disclosed herein may be formed from a wide variety of materials, unless described otherwise herein.

For purposes of this disclosure, the term “coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated.

It is also important to note that the construction and arrangement of the elements of the disclosure, as shown in the exemplary embodiments, are illustrative only. Although only a few embodiments of the present innovations have been described in detail in this disclosure, those skilled in the art who review this disclosure will readily appreciate that many modifications are possible (e.g., variations in sizes, dimensions, structures, shapes, and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

What is claimed is:

1. A temperature regulating and microclimate management system for a patient support apparatus, comprising:
a surface assembly including an outer covering having an inlet port and an outlet port and a spacer material disposed within an interior of the outer covering;
a pneumatic assembly in fluid communication with the inlet port, wherein the pneumatic assembly includes a housing and a blower disposed within the housing, and wherein the blower is configured to direct air through the spacer material via the inlet port; and
a controller communicatively coupled to the pneumatic assembly, wherein the controller is configured to activate the blower at a first operating speed to direct the air through the surface assembly to generate a cooling effect and configured to generate a heating effect through the surface assembly by at least one of:
adjusting the blower to a higher second operating speed to generate heat in the housing and warm the air, wherein the blower is configured to intake heated air and direct the heated air through the surface assembly;
restricting a discharge flow rate of the blower via a flow rate valve to generate the heat in the housing and direct the heated air through the surface assembly; and
recirculating exiting heated air from the outlet port to the pneumatic assembly to be redirected through the surface assembly.

2. The temperature regulating and microclimate management system of claim 1, wherein the controller is configured to generate the heating effect by both adjusting the blower to the second higher operating speed and restricting the discharge flow rate of the blower.

3. The temperature regulating and microclimate management system of claim 1, wherein the blower is configured to draw air along a height of the blower to warm the air with the heat to produce the heated air.

4. The temperature regulating and microclimate management system of claim 1, further comprising:
a switch valve in fluid communication with the pneumatic assembly and the surface assembly, wherein the switch valve is configured to exhaust the exiting heated air from the surface assembly when in an opened state in a cooling mode and recirculate the exiting heated air when in a closed state in a heating mode.

5. The temperature regulating and microclimate management system of claim 1, further comprising:
a sensor assembly communicatively coupled to the controller, wherein the controller is configured to adjust at least one of the blower and the flow rate valve in response to sensed information from the sensor assembly.

6. The temperature regulating and microclimate management system of claim 5, wherein the sensor assembly includes a thermal sensor and the sensed information includes a temperature of discharge air from the blower.

7. The temperature regulating and microclimate management surface of claim 1, further comprising:
a resistive heater communicatively coupled to the controller, wherein the resistive heater is configured to generate the heat to warm discharge air from the blower.

8. The temperature regulating and microclimate management system of claim 1, further comprising:
a frame assembly, wherein the pneumatic assembly is coupled to the frame assembly, and wherein the surface assembly is positioned on the frame assembly.

9. The temperature regulating and microclimate management system of claim 1, further comprising:
a blanket fluidly coupled with the pneumatic assembly, wherein the controller is configured to:
direct the air through the blanket and generate the cooling effect; and
generate the heating effect through the blanket.

10. A heating and cooling surface system, comprising:
a surface assembly including an outer covering and a spacer material disposed within an interior of the outer covering;
a pneumatic assembly in fluid communication with the interior, wherein the pneumatic assembly includes:
a housing;
a blower disposed within the housing, wherein the blower has an intake and a discharge outlet; and
a flow rate valve operably coupled to the discharge outlet of the blower, wherein the flow rate valve is configured to adjust a discharge flow rate of the blower; and
a controller communicatively coupled to the blower and the flow rate valve, wherein the controller is configured to:
adjust the flow rate valve to a first restriction setting for a cooling effect with an airflow directed through the surface assembly by the blower; and
adjust the flow rate valve to a second restriction setting for a heated airflow directed through the surface assembly by the blower for a heating effect, wherein the second restriction setting is higher than the first restriction setting to cause a greater restriction on the discharge flow rate from the blower to generate heat that is captured by the blower and produce the heating effect with the heated airflow.

11. The heating and cooling surface system of claim 10, wherein the controller is configured to adjust the blower to a first operating speed when the flow rate valve is at the first restriction setting and to a second higher operating speed when the flow rate valve is at the second restriction setting.

12. The heating and cooling surface system of claim 10, wherein an inlet opening of the housing is defined proximate to a base of the blower, and wherein the intake of the blower is defined at a top of the blower, and wherein the blower is configured to draw air from the inlet opening and along a height of the blower to capture the heat generated by the blower and warm the air to be directed through the surface assembly as the heated airflow for the heating effect.

13. The heating and cooling surface system of claim 10, further comprising:
a thermal sensor communicatively coupled to the controller, wherein the controller is configured to adjust the flow rate valve in response to sensed temperature of discharge air of the blower received from the thermal sensor.

14. The heating and cooling surface system of claim 13, further comprising:
a heater communicatively coupled to the controller, wherein the controller is configured to selectively activate the heater to increase a temperature of the discharge air from the blower in response to the sensed temperature from the thermal sensor being below a predefined temperature, and wherein the controller is configured to selectively deactivate the heater to decrease the temperature of the discharge air in response to the sensed temperature from the thermal sensor being above a predefined temperature.

15. A heating and cooling surface system, comprising:
a surface assembly including:
an outer covering; and
a spacer material disposed within an interior of the outer covering;
a pneumatic assembly in fluid communication with the interior, wherein the pneumatic assembly includes:
an enclosure operably coupled to the interior of the outer covering via inlet tubing and recirculation tubing;
a blower disposed within the enclosure, wherein the blower is configured to direct an airflow through the spacer material; and
a switch valve operably coupled to the recirculation tubing and an interior of the enclosure; and
a controller communicatively coupled to the blower and the switch valve, wherein the controller is configured to:
activate the blower at a first operating speed to generate a cooling effect with the airflow through the spacer material for a cooling mode;
activate the blower at a second operating speed to generate a heating effect with the airflow through the spacer material for a heating mode, the second operating speed being greater than the second operating speed to generate heat captured within the airflow; and
adjust the switch valve to a closed state in the heating mode to redirect the airflow from the surface assembly to the blower.

16. The heating and cooling surface system of claim 15, wherein the controller is configured to adjust the switch valve to an opened state to exhaust air from the surface assembly when in the cooling mode.

17. The heating and cooling surface system of claim 15, wherein the pneumatic enclosure includes a flow rate valve operably coupled with a discharge outlet of the blower, and wherein the controller is configured to:
adjust the flow rate valve to a first restriction setting in the cooling mode; and
adjust the flow rate valve to a second restriction setting in the heating mode, the second restriction setting being greater than the first restriction setting to reduce a discharge flow rate of discharge air from the blower and, consequently, cause the heat to be generated for the heating effect.

18. The heating and cooling surface system of claim 17, further comprising:
a thermal sensor communicatively coupled to the controller, wherein the controller is configured to adjust the flow rate valve in response to sensed temperature of discharge air of the blower received from the thermal sensor.

19. The heating and cooling surface system of claim 18, further comprising:
a heater communicatively coupled to the controller, wherein the controller is configured to selectively activate the heater to increase a temperature of the discharge air from the blower in response to the sensed temperature from the thermal sensor being below a predefined temperature, and wherein the controller is configured to selectively deactivate the heater to decrease the temperature of the discharge air in response to the sensed temperature from the thermal sensor being above a predefined temperature.

20. The heating and cooling surface system of claim 15, wherein the blower includes an electrical motor, and wherein more electrical energy is provided to the electrical motor in the heating mode to generate the heat compared to the electrical energy provided to the electrical motor in the cooling mode.
